SEQUENCE LISTING (14 sequences)

SEQ ID NO: 1 - DNA, 18 bp, Artificial
  18S forward primer
tagagggacaagtggcgt

SEQ ID NO: 2 - DNA, 18 bp, Artificial
  18S reverse primer
aatggggttcaacgggtt

SEQ ID NO: 3 - DNA, 20 bp, Artificial
  ACTA2 forward primer
cagccaggatgtgtgaagaa

SEQ ID NO: 4 - DNA, 20 bp, Artificial
  ACTA2 reverse primer
tcaccccctgatgtctagga

SEQ ID NO: 5 - DNA, 20 bp, Artificial
  Osteocalcin forward primer
ctccagccacaacatccttt

SEQ ID NO: 6 - DNA, 20 bp, Artificial
  Osteocalcin reverse primer
tggcctccagcactgtttat

SEQ ID NO: 7 - DNA, 20 bp, Artificial
  PECAM1 forward primer
atctgcatctcgtgggaagt

SEQ ID NO: 8 - DNA, 20 bp, Artificial
  PECAM1 reverse primer
gagctgaagtgtcagcagga

SEQ ID NO: 9 - DNA, 20 bp, Artificial
  RUNX2 forward primer
gcactacccagccaccttta

SEQ ID NO: 10 - DNA, 20 bp, Artificial
  RUNX2 reverse primer
tatggagtgctgctggtctg

SEQ ID NO: 11 - DNA, 20 bp, Artificial
  SNA1 forward primer
gcccaactacagcgagctac

SEQ ID NO: 12 - DNA, 20 bp, Artificial
  SNA1 reverse primer
ccaggagagagtcccagatg

SEQ ID NO: 13 - DNA, 20 bp, Artificial
  CDH5 forward primer
cgtggtggaaacacaagatg

SEQ ID NO: 14 - DNA, 20 bp, Artificial
  CDH5 forward primer
tgtgtacctggtctgggtga

(19) United States
(12) Patent Application Publication
(10) Pub. No.: US 2024/0216325 A1
(43) Pub. Date: Jul. 4, 2024

(54) NUCLEAR FACTOR KAPPA B PATHWAY INHIBITION TO ARREST CALCIFIC AORTIC STENOSIS

(71) Applicant: CORNELL UNIVERSITY, Ithaca, NY (US)

(72) Inventors: Jonathan BUTCHER, Ithaca, NY (US); Abulajiang MAIHEMUTI, Ithaca, NY (US); Terrence GEE, Laguna Niguel, CA (US)

(21) Appl. No.: 17/779,472

(22) PCT Filed: Nov. 25, 2020
(86) PCT No.: PCT/US2020/062416

§ 371 Date: May 24, 2022

Related U.S. Application Data
(60) Provisional application No. 62/940,131, filed on Nov. 25, 2019.

(51) Int. Cl. A61K 31/277; A61K 31/381; A61K 31/519; A61P 9/10

(52) U.S. Cl. CPC A61K 31/277; A61K 31/381; A61K 31/519; A61P 9/10

(57) ABSTRACT

The present application relates to a method for inhibiting calcification of aortic valves or inhibiting aortic valve sclerosis/fibrosis. The method involves selecting a subject having, or at risk of developing, calcific aortic valve disease or aortic valve sclerosis/fibrosis and administering, to the selected subject, an inhibitor of the nuclear factor kappa B (NFKB) signaling pathway to inhibit calcification of the selected subject's aortic valves or aortic valve sclerosis/fibrosis.

This application claims the priority benefit of U.S. Provisional Patent Application Ser. No. 62/940,131, filed Nov. 25, 2019, which is hereby incorporated by reference in its entirety.

This invention was made with government support under contract No. HL143247 awarded by the National Institutes of Health. The government has certain rights in the invention.

### FIELD

The present application relates to nuclear factor kappa b pathway inhibition to arrest calcific aortic stenosis.

### BACKGROUND

Calcific aortic valve disease (CAVD) is a serious and increasingly prevalent pathology, estimated to affect over 500 million people worldwide by 2050 (d'Arcy et al., “Large-scale Community Echocardiographic Screening Reveals a Major Burden of Undiagnosed Valvular Heart Disease in Older People: the OxVALVE Population Cohort Study,” Eur Heart J. 37:3515-3522 (2016)). CAVD is well recognized as an actively regulated cellular process (Mohler et al., “Bone Formation and Inflammation in Cardiac Valves,” Circulation 103:1522-1528 (2001)) characterized by progressive tissue fibrosis and obstructive mineralization causing aortic stenosis, hypertension, and congestive heart failure if not treated. CAVD often has a multi-year progression that affords biological intervention, but clinical trials employing vascular biology-derived biomarkers and therapeutics (e.g., statins) in CAVD have been unsuccessful (Cowell et al., “Scottish Aortic Stenosis and Lipid Lowering Trial, Impact on Regression (SALTIRE) Investigators. A Randomized Trial of Intensive Lipid Lowering Therapy in Calcific Aortic Stenosis,” N Engl J Med. 352:2389-2397 (2005); Otto, “Calcific Aortic Stenosis-Time to Look More Closely at the Valve,” N Engl J Med. 359:1395-1398 (2008); Rossebø et al., “SEAS Investigators. Intensive Lipid Lowering with Simvastatin and Ezetimibe in Aortic Stenosis,” N Engl J Med. 359:1343-1356 (2008)). There remains a significant need to establish valve-specific biological mechanisms regulating tissue homeostasis and calcific degeneration. Much of what is known about CAVD pathogenesis focuses exclusively on the valve interstitial cells (VIC). The aortic valve is also populated by a unique endothelial (valve endothelial cells [VEC]) phenotype, which must collaborate together with VIC to maintain phenotype quiescence within the demanding 3-dimensional (3D) microenvironment in which they reside (Towler, “Molecular and Cellular Aspects of Calcific Aortic Valve Disease,” Circ Res 113:198-208 (2013)). Very little is known about how each valve cell type engages valvular disease risk factors nor whether they progress mechanistically along the same molecular/cellular pathway.

Longitudinal PET/CT imaging indicates that inflammation of the valve both precedes calcification (Abdelbaky et al., “Early Aortic Valve Inflammation Precedes Calcification: a Longitudinal FDG-PET/CT Study,” Atherosclerosis 238:165-172 (2015)) and progresses proportionally with severity (Hjortnaes et al., “Arterial and Aortic Valve Calcification Inversely Correlates with Osteoporotic Bone Remodelling: a Role for Inflammation,” Eur Heart J. 31:1975-1984 (2010)). Inflammation has multifaceted origins and signaling pathways and is also caused by many other conditions that are often shared by patients with CAVD (e.g., renal disease, oxidative stress, hypertension) (Li et al., “The Progression of Calcific Aortic Valve Disease Through Injury, Cell Dysfunction, and Disruptive Biologic and Physical Force Feedback Loops,” Cardiovasc Pathol 22:1-8 (2013)). Recent work has shown the valve endothelium to be a novel source of oxidative stress driving myofibroblastic activation (Farrar et al., “Endothelial-derived Oxidative Stress Drives Myofibroblastic Activation and Calcification of the Aortic Valve,” PLoS One 10(4):e0123257 (2015)), and that inflammatory signaling can drive reactivation of embryonic-like endothelial to mesenchymal transformation (EndMT) as a potential mechanism of early valve dysfunction (Mahler et al., “Inflammatory Cytokines Promote Mesenchymal Transformation in Embryonic and Adult Valve Endothelial Cells,” Arterioscler Thromb Vasc Biol. 33:121-130 (2013)). However, it is unknown how specific inflammatory signaling pathways regulate unique and coordinated activities in these two cell populations toward CAVD initiation or downstream calcific progression.

NFκB (nuclear factor κ-light-chain enhancer of activated B cells) is a prominent transcription factor family that is involved in many cardiovascular pathologies (Tak et al., “NF-kappaB: a Key Role in Inflammatory Diseases,” J Clin Invest. 107:7-11 (2001); Van der Heiden et al., “Role of Nuclear Factor KappaB in Cardiovascular Health and Disease,” Clin Sci (Lond) 118:593-605 (2010)). Activated NFκB subunits, including p65/RelA, translocate to the nucleus and directly activate gene transcription controlling cell proliferation, survival, immune, or differentiation responses (Hoffmann et al., “Circuitry of Nuclear Factor KappaB Signaling,” Immunol Rev. 210:171-186 (2006)). Conversely, NFκB can be sequestered in the cytoplasm via binding to IkBs, such as IκBa (nuclear factor of kappa light polypeptide gene enhancer in B-cells inhibitor, alpha). The IKK signaling complex is a key convergence site for multiple signaling pathways leading to NFκB activation, with IKKβ (inhibitor of nuclear factor kappa-B kinase subunit beta) participating in the primary pathway of proinflammatory induction (Tak et al., “NF-kappaB: a Key Role in Inflammatory Diseases,” J Clin Invest. 107:7-11 (2001)). Recent evidence implicates TNFα (tumor necrosis factor α) inflammatory signaling in the induction of EndMT in aortic VEC in vitro (Mahler et al., “Inflammatory Cytokines Promote Mesenchymal Transformation in Embryonic and Adult Valve Endothelial Cells,” Arterioscler Thromb Vasc Biol. 33:121-130 (2013); Farrar et al., “Heterogeneous Susceptibility of Valve Endothelial Cells to Mesenchymal Transformation in Response to TNFα,” Ann Biomed Eng. 42:149-161 (2014)). TNFα has also been shown to accelerate VIC calcification (Yu et al., “Tumor Necrosis Factor-alpha Accelerates the Calcification of Human Aortic Valve Interstitial Cells Obtained From Patients with Calcific Aortic Valve Stenosis via the BMP2-Dlx5,” J Pharmacol Exp Ther. 337:16-23 (2011)) with NFκB and has also been associated with osteoclast and bone remodeling activity (Crotti et al., “Factors Regulating Osteoclast Formation in Human Tissues Adjacent to Pen-implant Bone Loss: Expression of Receptor Activator NFkappaB, RANK Ligand and Osteoprotegerin,” Biomaterials 25:565-573 (2004)).

The present application is directed to overcoming these and other deficiencies in the art.

### SUMMARY

A first aspect of the present application relates to a method for inhibiting calcification of aortic valves or inhibiting aortic valve sclerosis/fibrosis. The method involves selecting a subject having, or at risk of developing, calcific aortic valve disease or aortic valve sclerosis/fibrosis and administering, to the selected subject, an inhibitor of the nuclear factor kappa B (NFκB) signaling pathway to inhibit calcification of the selected subject's aortic valves or aortic valve sclerosis/fibrosis.

A second aspect of the present application relates to a method for inhibiting myofibroblastic and osteogenic differentiation of aortic valvular interstitial cells and/or aortic valvular endothelial cells. The method involves administering, to the aortic valvular interstitial cells and/or aortic valvular endothelial cells, an inhibitor of the nuclear factor kappa B (NFκB) signaling pathway to inhibit myofibroblastic and osteogenic differentiation of the cells.

In the present application, it is identified how RelA specific NFκB activity drives VEC and VIC NFκB pathogenic mechanisms in 3D culture. Valve lineage-specific Cre mice are further employed to test the role of altered p65/RelA NFκB activity in valve cells in normal or established diet-induced hypercholesterolemic CAVD animal models. Additionally, lineage tracing is employed to map the fate of VECs in these conditions. The findings establish that activation of NFκB is sufficient to induce calcific degenerative processes in both VEC and VIC in vitro and in vivo. Furthermore, genetic inhibition of NFκB activation in valves in vivo mitigates aortic valve-specific calcific progression in established CAVD environments. These findings establish that CAVD involves multicellular engagement through diverse cellular events that are controlled at least in part by a shared molecular pathway via p65/RelA.

### BRIEF DESCRIPTION OF THE DRAWINGS

FIG. 1A shows Russell-Movat staining of non-calcified, sclerotic, and calcified/stenosed human aortic valves. Scale bar is 200 um. FIG. 1B shows sister section immunofluorescence stain for NFkB p65 and CD31. Letters indicate trilaminar region classifications of valve for subsequent analysis (v: ventricularis, s: spongiosa, f: fibrosa). Scale bar is 200 um. FIG. 1C shows macroscopic images of fixed non-calcified, sclerotic, and calcified/stenosed human aortic valves. Scale bar is 1 cm.

FIG. 2A shows representative images of CD31 and NFκB (nuclear factor κ-light-chain enhancer of activated B cells) p65 costaining of noncalcified, sclerotic, and calcified/stenosed human aortic valves (horizontal axis). In FIG. 2A, panels indicate expression at ventricularis, spongiosa, and fibrosa regions (vertical axis). White arrows indicate nuclear p65 expression. Asterisks indicate neo-vessel formation in intimal region. Yellow arrows indicate CD31-positive cells in the subendothelial region, independent of neo-vessel formation. Scale bar is 100 μm. FIG. 2B shows nuclear p65 integrated density, normalized to the sclerotic valve ventricularis expression. Statistical significance indicated comparing different patient groupings for the same valvular-region (e.g., ventricularis of noncalcified vs sclerotic vs calcified valves; *P<0.05; **P<0.005, ***P<0.0005).

FIG. 3A shows immunofluorescence staining of IKKβ expression of 5 month-old WT and IKKβ^{valveKO} mouse valves, scale bar is 20 um. FIG. 3B shows Russell-Movat staining of 5 month-old WT and IKKβ^{valveKO} (Nfatc1^{Cre};IKKβ^{flox/flox}) mouse hearts, magnified at aortic valve region. Scale bar is 0.5 mm. FIGS. 3C-3E show echocardiographic measurements of peak velocity, peak gradient, and mean gradient through aortic valve, no difference between groups (n>5). FIG. 3F shows measurement of aortic root diameter from echocardiographic M-mode with comparison between systolic and diastolic ratio, no difference between groups (n>5). FIG. 3G shows thickness of the AoV leaflet, measured from EKV. No difference between groups. (n>5). Error bars indicate SEM.

FIG. 4A shows representative images of NFκB (nuclear factor κ-light-chain enhancer of activated B cells) p65/RelA protein expression and nuclear localization (arrows) in aortic valve leaflets of 5-mo-old wild-type (WT), AVD (aortic valve disease), and AVD+IKKβ^{f/f} (inhibitor of nuclear factor kappa-B kinase subunit beta) mice. Scale bars are 100 um (left-most column) and 20 m (second column on). FIG. 4B shows representative images of VCAM1 (vascular cell adhesion molecule 1) protein expression from same groups. Scale bar is 100 μm. FIG. 4C shows TUNEL (terminal deoxynucleotidyl transferase dUTP nick-end labeling) assay for apoptosis in aortic valve (AoV) of same groups, quantification of the percent of cells in the AoV positive for TUNEL staining (n=5). Scale bar is 100 μm. FIG. 4D shows analysis of proliferation in AoV of same groups via Ki67 staining. Ki 67 (green) and CD31 (red), scale bar is 100 μm. FIGS. 4E-4F show quantification of percent of cells in the AoV positive for nuclear Ki67 (n=5). LDLR indicates low-density lipoprotein receptor. Asterisks indicate statistically significant groups (*P<0.05).

FIGS. 5A-5F show echocardiographic measurements of aortic valve (AoV) function of 5-mo-old mice, 4 mo of high-fat diet in all models except wild-type (WT) control (WT: n=11 LDLR [low density lipoprotein receptor]^{-/-}: n=17, LDLR^{-/-} IKKB^{valveKO}: n=11). FIGS. 5A, 5B, and SC show mean AoV peak jet velocity (mm/s), peak gradient (mm Hg), and mean gradient (mm Hg) from pulsed-wave Doppler imaging, calculated using the average peak from >3 cardiac cycles for each mouse. FIG. 5D shows a ratio of aortic root systolic:diastolic ratio calculated from M-mode echocardiographic measurements of aortic root diameter during peak diastole and systole.

FIG. 5E shows cardiac ejection fraction percentage calculated from parasternal long-axis ventricular volume at peak systole and diastole. Asterisks indicate statistical significance, *P<0.05, **P<0.005, ***P<0.0005. FIG. 5F shows aortic leaflet thickness measurements.

FIGS. 6A-6B shows comparison of Sex-specific echocardiographic parameters of transgenic mouse lines. (WT: n=6 male, 5 female LDLR−/−: n=10 male 7 female, LDLR−/− IKKBvalveKO: n=6 male 5 female).

FIG. 7A shows Alizarin Red S (ARS) stain for calcium in aortic valve (AoV). Scale bar is 100 μm. FIG. 7B shows von Kossa staining for mineral deposition in AoV. Scale bar is 100 μm. FIG. 7C shows Russell-Movat stain for collagen (yellow), glycosaminoglycans (blue), elastin (black), and mucins (red) in AoV. Quantification indicates percent of the total valve area that was positive for indicated stain, measured from valve hinge-point between annulus and cusp to the distal tip (n=5). LDLR indicates low-density lipoprotein receptor; and WT, wild-type. Asterisks indicate statistical significance (*P<0.05, **P<0.005, ***P<0.0005, ****P<0.00005).

FIG. 8 shows glycosaminoglycan (GAG) content according to Russell-Movat stain (blue). n=5.

FIG. 9A shows Alizarin Red S (ARS) staining for calcium in 14-d valve endothelial cells (VEC) treated with basal growth medium (GM), osteogenic differentiation medium (OGM), OGM+TNFα (tumor necrosis factor α; 30 ng/mL), or OGM+p65 overexpression. Scale bar is 100 μm. FIG. 9B shows quantification of calcium deposition using ARS dye-extraction assay, =normalized to GM+ empty vector control (n=6). *Significant from groups 1, 2, #significant from group 3; P<0.05. FIG. 9C shows αSMA (alpha-smooth muscle actin) protein expression in VEC with or without p65 overexpression with 14-d control or OGM treatment. Scale bar is 20 μm. FIG. 9D shows ACTA2 (αSMA gene) mRNA in VEC, same conditions as 9C (n=4). FIG. 9E shows Runx2 (runt-related transcription factor 2) protein expression in VEC, same conditions as 9C. Scale bar is 20 μm. FIG. 9F shows Runx2 mRNA in VEC, same conditions as 9E (n=4). FIG. 9G shows OCN (osteocalcin) protein expression in VEC, same conditions as 9C. FIG. 9H shows OCN mRNA in VEC, same conditions as 9G. Statistical significance between different groups are indicated by unshared letter assignments, P<0.05. Different letters indicate statistically significant differences between groups (P<0.05). Error bars indicate SEM.

FIG. 10A shows representative images of p65 expression in 48 hour VEC on 3D hydrogels treated with TNFα or OGM. White arrows indicate cells with nuclear-positive signal. Scale bar is 20 μm. FIG. OB shows quantification of p65 nuclear translocation in VEC using the ratio of nuclear to cytosolic p65 protein (nuc:cyto ratio). n=4. Asterisks indicate statistically significant groups (*: p<0.05).

FIG. 11A shows p65 expression in 14-day VIC in 3D hydrogels treated with CTL or OGM. Scale bar is 20 μm. FIG. 11B shows quantification of p65 nuclear translocation in VIC using the ratio of nuclear to cytosolic p65 protein (nuc:cyto ratio). n=4.

FIG. 12A shows Alizarin Red S (ARS) staining for calcium in 14-d valve interstitial cell (VIC) with p65 overexpression (+p65) or inhibition (+IκBα) treated with basal growth medium (GM) or osteogenic differentiation medium (OGM). Scale bar is 100 μm. FIG. 12B shows quantification of calcium deposition using ARS dye-extraction assay, normalized to GM+ empty vector control (n=6). *Significant from groups 1, 3, 5; #significant from 2; $significant from 4, P<0.05. FIG. 12C shows αSMA (alpha-smooth muscle actin) protein expression in VIC with p65 overexpression (+p65) or inhibition (+IκBα) vectors, with 14-d GM or OGM treatment. Scale bar is 20 m. FIG. 12D shows ACTA2 (αSMA gene) mRNA in VIC, same conditions as in FIG. 12C (n=4). FIG. 12E shows Runx2 (runt-related transcription factor 2) protein expression in valve endothelial cells, same conditions as FIG. 12C. Scale bar is 20 μm. FIG. 12F shows Runx2 mRNA in VIC, same conditions as FIG. 12E (n=4). FIG. 12G shows OCN (osteocalcin) protein expression in VIC, same conditions as FIG. 12E. FIG. 12H shows OCN mRNA in VIC, same conditions as FIG. 12G (n=4). Statistical significance between different groups is indicated by unshared letter assignments. Different letters indicate statistically significant differences between groups (P<0.05). Error bars indicate SEM. A.U. indicates absorbance units.

FIG. 13A shows VE-cad (vascular endothelial cadherin) protein expression in valve endothelial cells (VEC) with p65 overexpression or inhibition (+IκBα), with 48 h basal growth medium (GM) or GM+TNFα (tumor necrosis factor α; 30 ng/mL) treatment. Scale bar is 20 μm. FIG. 13B shows CDH5 (VE-cad gene) mRNA in VEC, same conditions as A (n=4). *Statistically significant from groups 1, 5, 6, P<0.05. FIG. 13C shows αSMA (alpha-smooth muscle actin) protein expression in VEC, same conditions as FIG. 13A. Scale bar is 20 m. FIG. 13D shows ACTA2 (αSMA gene) mRNA in VEC, same conditions as FIG. 13A (n=4). *Statistically significant from group 1, P<0.05, #statistically significant from all other groups, P<0.05. FIG. 13E shows SNAI1 (snail family transcriptional repressor 1) mRNA in VEC, same conditions as FIG. 13A (n=4). *Statistically significant from group 1, P<0.05, #statistically significant from all other groups, P<0.05. FIG. 13F shows CD31 and αSMA expression in 5 mo-old mice AoVs. Arrows indicate cells coexpressing CD31 and αSMA. Scale bar is 100 μm. FIG. 13G shows β-Galactosidase staining with and Nuclear Fast Red counterstaining in Nfatc1enCreRosa26lacZ reporter mice in either the wild-type (WT) or CAVD (calcific aortic valve disease) background. Asterisks indicate β-gal-positive cells in interstitial space. Scale bar is 100 μm. FIG. 13H shows Alizarin Red S (ARS) staining of calcium deposition. Asterisks indicate β-gal-positive cells in interstitial space. Scale bar is 100 m. CTL indicates control; GM, basal growth medium; and LDLR, low-density lipoprotein receptor.

FIGS. 14A-14G show echocardiographic measurements of aortic valve (AoV) function of 5-mo old mice, 4 mo of high-fat diet in all models except wild-type (WT) control (WT: n=11, LDLR [low-density lipoprotein receptor]^{-/-}: n=17, LDLR^{-/-} IKKB^{vecKO}: n=15). FIGS. 14A, 14B, and 14C show the mean AoV peak jet velocity (mm/s), Peak gradient (mm Hg), and mean gradient (mm Hg) from pulsed-wave Doppler imaging, calculated using the average peak from >3 cardiac cycles for each mouse. FIG. 14D shows the ratio of aortic root systolic:diastolic ratio calculated from M-mode echocardiographic measurements of aortic root diameter during peak diastole and systole. FIG. 14E shows the cardiac ejection fraction percentage calculated from parasternal long-axis ventricular volume at peak systole and diastole. Asterisks indicate statistical significance (*P<0.05, FIG. 14F shows Alizarin Red S (ARS) staining for calcium in AoV. Scale bar is 100 μm. FIG. 14G shows von Kossa staining for mineral deposition in AoV. Scale bar is 100 μm. Quantification indicates percent of the total valve area that was positive for indicated stain, measured from valve hinge-point between annulus and cusp to the distal tip (n=5). Asterisks indicate statistical significance (*P<0.05, **P<0.005. ***P<0.0005, ****P<0.00005).

FIGS. 15A-15B show comparison of Sex-specific echocardiographic parameters of transgenic mouse lines. (WT: n=6 male, 5 female LDLR−/−: n=10 male 7 female, LDLR−/− IKKBvalveKO: n=6 male 9 female).

FIG. 16 shows total serum cholesterol in each group, collected at 5 months of age (n=5). Groups that do not share letters are statistically significant, p<0.05. Error bars indicate SEM.

FIGS. 17A-17C show the effects of blocking the NFκB pathway in a mouse model of CAVD.

FIGS. 18A-18C show hemodynamic changes following treatment with Bay11-7082.

FIGS. 19A-19C show hemodynamic changes following treatment with BH4.

FIGS. 20A-20C show histology staining of the aortic valve calcium content.

FIGS. 21A-21D show mRNA expression of fibro-osteogenic gene profiles.

FIGS. 22A-22D show the effects of blocking the NFκB pathway using BAY11-7082 pharmacological treatment on an in vitro valve calcification model.

FIGS. 23A-23C show the effects of blocking the NFκB pathway using TPCA-1 pharmacological treatment on an in vitro valve calcification model

### DETAILED DESCRIPTION

The present application relates to a method for inhibiting calcification of aortic valves or inhibiting aortic valve sclerosis/fibrosis. The method involves selecting a subject having, or at risk of developing, calcific aortic valve disease or aortic valve sclerosis/fibrosis and administering, to the selected subject, an inhibitor of the nuclear factor kappa B (NFκB) signaling pathway to inhibit calcification of the selected subject's aortic valves or aortic valve sclerosis/fibrosis.

Calcific aortic valve disease is the most common heart valve disease in the Western World and the third cause of cardiovascular disease after coronary artery disease and systemic arterial hypertension (Donato et al., “Current Evidence and Future Perspectives on Pharmacological Treatment of Calcific Aortic Valve Stenosis,” Int. J. Mol. Sci. 21(21):8263 (2020), which is hereby incorporated by reference in its entirety). The prevalence increases with advancing age, reaching 12% in the elderly (>75 years) (Donato et al., “Current Evidence and Future Perspectives on Pharmacological Treatment of Calcific Aortic Valve Stenosis,” Int. J. Mol. Sci. 21(21):8263 (2020), which is hereby incorporated by reference in its entirety).

In accordance with all aspects of the present application, a “subject” encompasses any animal including a fish, bird, or reptile, or a mammal, e.g., human, rabbit, cow, pig, sheep, chicken, rat, or mouse.

As used herein, the term “aortic valve” refers to the heart valve that divides the left ventricle and the aorta. The aortic valve opens during left ventricular contraction and then closes to prohibit the backwash of oxygenated blood from the aorta into the ventricle. The aortic valve typically contains 3 valve leaflets in most individuals, but may contain 2 valve leaflets in some individuals.

As used herein, “aortic valve disease” refers to a disease state in which there is calcification or fibrosis of the aortic valve, encompassing aortic sclerosis and aortic stenosis.

As used herein, “aortic sclerosis” refers to the thickening of the aortic valve leaflets in the absence of obstruction to left ventricular outflow.

As used herein, “aortic stenosis” refers to a condition of valvular pathology in which left ventricular outflow is obstructed or narrowed, either statically or dynamically. The net effect is an increase in pressure drop across the aortic root and/or increased blood velocity from left ventricular ejection.

As used herein, “inhibiting” refers to the reduction or suppression of a given condition, symptom, disorder, or disease, or a decrease in the baseline activity of a biological activity or process.

As used herein, “an inhibitor of the nuclear factor kappa B (NFκB) signaling pathway” means a compound that inhibits, directly or indirectly, any step in the cell transcription factor nuclear kappa-B (NFκB) signaling pathway.

Subjects having calcific aortic valve disease or aortic valve sclerosis/fibrosis may have any stage/phase of the disease. CAVD is a multi-step disease that can be divided into two distinct phases: an early initiation phase and a later propagation phase, each characterized by different mechanisms. The initiation phase, termed aortic sclerosis, shows similarities with atherosclerosis. The initiating event is represented by an endothelial damage on the aortic side of the valve, due to increased mechanical stress and reduced shear stress. A series of events involving cell infiltration and lipoproteins promotes inflammation and lipid accumulation. The release of pro-inflammatory cytokine induced further oxidative stress and inflammatory response. The second phase of the disease, the propagation phase, is characterized by fibrosis and calcification. In this phase, the valvular interstitial cells (VICs) are activated, which leads to increased production and deposition of extracellular matrix components such as collagen fibers. This results in fibrotic remodeling of the tissue. Valvular calcification also occurs in this phase.

In one embodiment, the selected subject has calcification of aortic valves.

In another embodiment, the selected subject has aortic valve sclerosis/fibrosis.

Subjects selected for the method described herein may also be at risk of developing calcific aortic valve disease or aortic valve sclerosis/fibrosis. Risk factors for developing calcific aortic valve disease or aortic valve sclerosis/fibrosis include, without limitation, being of age 65 and older, male gender, cigarette smoking, hypertension, dyslipidemia, metabolic syndrome, diabetes, being overweight or obese, kidney dysfunction and mineral metabolism, family history of aortic stenosis, elevated lipoprotein(a), and congenital cardiac malformations (Chen et al., “Risk Factors for Valvular Calcification,” Curr. Opin. Endocrinol. Diabetes Obes. 26(2):96-102 (2019), which is hereby incorporated by reference in its entirety).

Those at risk for heart valve disease are also included in the methods described herein. Such subjects include those with a genetic/epigenetic predisposition, family history, and/or presence of comorbidities known to increase risk for heart valve disease. Genetic predispositions include mono-genetic mutations such as known with Notch1 (Garg et al., “Mutations in NOTCH1 Cause Aortic Valve Disease,” Nature 437(7056):270-4 (2005), which is hereby incorporated by reference in its entirety) or Gata6 (Gharibeh et al., “GATA6 Regulates Aortic Valve Remodeling, and its Haploinsufficiency Leads to Right-Left Type Bicuspid Aortic Valve,” Circulation 138(10):1025-1038 (2018), which is hereby incorporated by reference in its entirety), deficient methylation states, microRNA mutations, and also include multi-genetic changes for example with exocyst-cilia machinery (Fulmer et al., “Defects in the Exocyst-Cilia Machinery Cause Bicuspid Aortic Valve Disease and Aortic Stenosis,” Circulation 140(16):1331-1341 (2019), which is hereby incorporated by reference in its entirety). Family history includes patients with polycystic kidney disease, bicuspid aortic valves, or hypercholesterolemia. Comorbidities that increase risk for heart valve disease include hypertension, obesity, coronary artery disease, and congenital heart disease. Heart valve disease is typically diagnosed via ultrasound echocardiography whereby elevated blood velocities and/or pressure gradients through the valve root are discovered. Often this is accompanied by a radiological scan (e.g. CT), which identifies the presence of calcium deposits in the aortic root complex, particularly on the leaflets. Patients with calcific heart valve disease are often monitored for disease progression, which provides a window of pharmacological therapy prior to a decision for prosthetic surgical replacement (the current only FDA approved therapy for heart valve disease).

Accordingly, in one embodiment, the selected subject is at risk of developing calcific aortic valve disease. In another embodiment, the selected subject is at risk of developing aortic valve sclerosis/fibrosis.

In accordance with the method described herein, an inhibitor of the nuclear factor kappa B (NFκB) signaling pathway is then administered to the selected subject to inhibit calcification of the selected subject's aortic valves or aortic valve sclerosis/fibrosis.

NFκB is a transcription factor that plays an important role in many cellular processes. Vertebrate NFκB transcription complexes can be any of a variety of homo- and heterodimers formed by the subunits p50, p52, c-Rel, RelA (p65), and RelB. In its inactive state, NFκB resides in the cytoplasm and is bound to another protein called IκB. Activation of the NFkB signaling pathway begins upon cell activation. IκB may be modified and targeted for degradation by IκB (IKK) kinase. The IKK complex contains two kinase subunits, IKKα and IKKβ, and an associated scaffold-like regulatory protein called NEMO (IKKγ). After stimulation of cells by agents such as tumor necrosis factor α (TNFα), interleukin-1 (IL-1) or various pathogens, the IKK complex is activated in part by phosphorylation of specific serine residues in the activation loop of each IKK subunit. The activated IKK complex can then phosphorylate IκB on two serine residues in human IκB. Phosphorylation of the IκB by IKK signals it for ubiquitination at specific lysine residues by the SCF-β-TrCP E3 ubiquitin ligase complex, which targets the IκB for degradation by the 26S proteasome The freed NFκB may then translocate into the nucleus, and along with other transcription factors, activate transcription of target genes (see e.g., Karin et al., “The IKK NF-kB System: a Treasure Trove for Drug Development,” Nat. Rev. 3:17-26 (2004); Gilmore et al., “Inhibitors of NF-kB Signaling: 785 and Counting,” Oncogene 25:6887-6899 (2006), which are hereby incorporated by reference in their entirety).

NFκB activation refers to a state of the NFκB molecule that is capable of participating in transcription activation. Inhibitors of NFκB activation generally refer to an agent that either partially or completely blocks NFκB participation in the activation of many its target genes. A large number of NFκB target genes have been reported in the literature. The mRNAs of these target genes are normally present at low levels and their levels increase dramatically when NFκB and other transcription factors bind to regulatory elements of these genes and activate their transcription.

In practicing the methods of the present application, the inhibitor of the NFκB signaling pathway may inhibit NFκB at any step in the NFκB activation process. The inhibitor may be a general inhibitor of NFκB activation, or the inhibitor may inhibit specific pathways of induction. Inhibitors that target multiple steps in the NFκB signaling pathway are also contemplated for use herein. Generally, inhibition of NF-κB activation can occur by three mechanisms: (1) blockage of the incoming activation signal at an early stage (e.g., binding of ligand to its receptor) resulting in complete abrogation of the signal's effect; (2) interference with a cytoplasmic step in the NFκB activation pathway by blockage of a specific component of the cascade (e.g., the activation of the IKK complex or degradation of IκB); or (3) blockage of NFκB nuclear activity, that is, inhibiting its translocation to the nucleus, its binding to DNA, a nuclear modification of NF-κB that affects its activity or specificity (e.g. protein acetylation and/or methylation), or an interaction of NFκB on DNA with specific or basal transcription machinery.

Thus, in practicing the methods of the present application, the inhibitor of the NFκB signaling pathway may be, without limitation, an upstream NFκB target inhibitor, an IKK phosphorylation inhibitor, an IκB phosphorylation inhibitor, an IκB degradation inhibitor, a proteasome inhibitor, a protease inhibitor, an IκB upregulation inhibitor, and NFκB nuclear translocation inhibitor, an NFκB expression inhibitor, and an NFκB transactivation inhibitor.

In one embodiment, the inhibitor of the NFκB signaling pathway is selected from the group consisting of a protein kinase inhibitor, a protein phosphatase inhibitor, an inhibitor of protein acetylation, a protein methyltransferase inhibitor, a proteasome inhibitor, an inhibitor of protein ubiquitination, an NFκB nuclear translocation inhibitor, an inhibitor of NFκB DNA binding activity.

The inhibitor of the NFκB signaling pathway may also be an inhibitor of eNOS uncoupling. eNOS is known in the art to prevent vascular inflammation and is regulated by NFκB. In various diseases, the function of endothelial nitric oxide (NO) synthase (eNOS) is altered, and it produces superoxide instead of NO. This alteration is referred to as “eNOS uncoupling” and is linked to an increased monomerization of the enzyme and endothelial dysfunction (Yang et al., “eNOS Uncoupling and Endothelial Dysfunction in Aged Vessels,” Am. J. Physiol. Heart Circ. Physiol. 297(5):H1829-H1836 (2009), which is hereby incorporated by reference in its entirety). Further, eNOS uncoupling causes Reactive Oxidative Stress (ROS) that drives NFκB activation as a transcriptional outcome (Farrar et al., “Endothelial-derived Oxidative Stress Drives Myofibroblastic Activation and Calcification of the Aortic Valve,” PLoS One 10(4):e0123257 (2015), which is hereby incorporated by reference in its entirety). Blocking ROS is another means to block NFκB activation if that is the inducer.

Specific inhibitors of the NFκB signaling pathway are well known in the art and include, without limitation, those identified in Gilmore et al., “Inhibitors of NF-kB Signaling: 785 and Counting,” Oncogene 25:6887-6899 (2006), which is hereby incorporated by reference in its entirety.

The inhibitor may be, without limitation, a small molecule, a peptide, a nucleic acid, an antioxidant, a microbial protein, a viral protein, or an anti-inflammatory agent.

Small molecule NFκB signaling pathway inhibitors are well known in the art and are described in, for example, Gilmore et al., “Inhibitors of NF-κB Signaling: 785 and Counting,” Oncogene 25:6887-6899 (2006) and Ramadass et al., “Small Molecule Nf-κB Pathway Inhibitors in Clinic,” Int. J. Mol. Sci. 21(14):5164 (2020), which are hereby incorporated by reference in their entirety. As used herein, “small molecules” are typically organic, peptide or non-peptide molecules, having a molecular weight less than 10,000 Da, less than 5,000 Da, less than 1,000 Da, and less than 500 Da. This class of modulators includes chemically synthesized molecules, for instance, compounds from combinatorial chemical libraries.

Peptides are also contemplated for use as inhibitors of the NFκB signaling pathway. For example, cell-permeable peptides, such as SN-50, that contain nuclear localization sequences of NFκB can saturate the process involved in the uptake of NFκB into the nucleus (Gilmore et al., “Inhibitors of NF-kB Signaling: 785 and Counting,” Oncogene 25:6887-6899 (2006), which is hereby incorporated by reference in its entirety).

The peptides used in conjunction with the present application can be obtained by known isolation and purification protocols from natural sources, can be synthesized by standard solid or solution phase peptide synthesis methods according to the known peptide sequence of the peptide, or can be obtained from commercially available preparations or peptide libraries. Included herein are peptides that exhibit the biological binding properties of the native peptide and retain the specific binding characteristics of the native peptide. Derivatives and analogs of the peptide, as used herein, include modifications in the composition, identity, and derivitization of the individual amino acids of the peptide provided that the peptide retains the specific binding properties of the native peptide. Examples of such modifications would include modification of any of the amino acids to include the D-stereoisomer, substitution in the aromatic side chain of an aromatic amino acid, derivitization of the amino or carboxyl groups in the side chains of an amino acid containing such a group in a side chain, substitutions in the amino or carboxy terminus of the peptide, linkage of the peptide to a second peptide or biologically active moiety, and cyclization of the peptide (G. Van Binst and D. Tourwe, “Backbone Modifications in Somatostatin Analogues: Relation Between Conformation and Activity,” Peptide Research 5:8-13 (1992), which is hereby incorporated by reference in its entirety).

Nucleic Acids may also be used to inhibit NFκB pathway activity. By way of example, specific NFκB DNA binding can be blocked by the use of decoy oligonucleotides that have NFκB binding sites. These decoy oligonucleotides function by competing with NFκB binding to specific gene promoters (Gilmore et al., “Inhibitors of NF-kB Signaling: 785 and Counting,” Oncogene 25:6887-6899 (2006), which is hereby incorporated by reference in its entirety).

Antioxidants are also contemplated for use in the methods described herein. Several antioxidants are known in the art to inhibit the NFκB signaling pathway and are described in Gilmore et al., “Inhibitors of NF-kB Signaling: 785 and Counting,” Oncogene 25:6887-6899 (2006), which is hereby incorporated by reference in its entirety.

Viruses can produce certain proteins that have mechanisms to inhibit NFκB signaling, including, without limitation, the encoding of IκB-like inhibitors of NFκB, the cleaving of p65, and the targeting of IKK (Powell et al., J Virol 70:8527-8533 (1996); Camus-Bouclainville et al., J Virol 78:2510-2516 (2004); Thoetkiattikul et al., Proc Natl Acad Sci USA 102:11426-11431 (2005), which are hereby incorporated by reference in their entirety). Therefore, use of such viral proteins is also contemplated.

Microbial proteins also produce proteins capable of inhibiting the NFκB signaling pathway. By way of example, the YopJ protein encoded by the enteropathogen Yersinia pseudotuberculosis inhibits NFκB activation by deubiquitinating IκB, which prevents its degradation (Gilmore et al., “Inhibitors of NF-kB Signaling: 785 and Counting,” Oncogene 25:6887-6899 (2006), which is hereby incorporated by reference in its entirety).

Anti-inflammatory agents are also contemplated for use as inhibitors of the NFκB signaling pathway in the methods herein. Anti-inflammatory agents that inhibit NFκB signaling are well known in the art. For example, nonsteroidal anti-inflammatory drugs, such as aspirin, ibuprofen, sulindac, indomethacin are known in the art to inhibit NFκB activation (Gilmore et al., “Inhibitors of NF-kB Signaling: 785 and Counting,” Oncogene 25:6887-6899 (2006), which is hereby incorporated by reference in its entirety). Glucocorticoids, such as dexamethasone, prednisone and methylprednisolone are also known to inhibit NFkB (Gilmore et al., “Inhibitors of NF-kB Signaling: 785 and Counting,” Oncogene 25:6887-6899 (2006), which is hereby incorporated by reference in its entirety).

In one embodiment, the inhibitor of the NFκB signaling pathway is selected from the group consisting of ATP analogs, BMS-34554, parthenolide, arsenite, epoxyquinoids, gene-based inhibitors, SB203580, denbinobin, tyrosine kinase inhibitors, rhein, TNAP, betaine, epoxyquinol B, M2L, CCK-8, KSR2, golli BG21, BAY11-7082, protein phosphatase 2A, cytosine arabinoside, OspF, gallic acid, Daxx, anacardic acid, Set9 inhibitor, bortezomib, ALLnL, LLM, Z-LLnV, Z-LLL, lactacystine, N-cbz-Leu-Leu-leucinal (MG132), MG115, ubiquitin ligase inhibitors, salinosporamide A (NPI-0052), DCIC, TPCK, TLCK, BTEE, APNE, YopJ, R0196-9920, A20 (TNFAIP3), SN50, dehydroxymethylepoxyquinomicin, sesquiterpene lactones, decoy oligodeoxynucleotides, BH4, TPCA-1, and combinations thereof.

In certain embodiments, the inhibitor of the NFκB signaling pathway is BAY11-7082.

In other embodiments, the inhibitor of the NFκB signaling pathway is BH4.

In practicing the methods of the present application, the administering step is carried out systemically or via direct or local administration. By way of example, suitable modes of systemic administration include, without limitation orally, topically, transdermally, parenterally, intradermally, intramuscularly, intraperitoneally, intravenously, subcutaneously, or by intranasal instillation, by intracavitary or intravesical instillation, intraocularly, intraarterialy, intralesionally, or by application to mucous membranes. Suitable modes of local administration include, without limitation, catheterization, implantation, direct injection, dermal/transdermal application, or portal vein administration to relevant tissues, or by any other local administration technique, method or procedure generally known in the art. The mode of affecting delivery of the inhibitor will vary depending on the type of inhibitor.

The inhibitor may be orally administered, for example, with an inert diluent, or with an assimilable edible carrier, or it may be enclosed in hard or soft shell capsules, or it may be compressed into tablets, or they may be incorporated directly with the food of the diet. The inhibitor may also be administered in a time release manner incorporated within such devices as time-release capsules or nanotubes. Such devices afford flexibility relative to time and dosage. For oral therapeutic administration, the inhibitor may be incorporated with excipients and used in the form of tablets, capsules, elixirs, suspensions, syrups, and the like. Such compositions and preparations should contain at least 0.1% of the inhibitor, although lower concentrations may be effective and indeed optimal. The percentage of the inhibitor in these compositions may, of course, be varied and may conveniently be between about 2% to about 60% of the weight of the unit.

When the inhibitor is administered parenterally, solutions or suspensions of the inhibitor can be prepared in water suitably mixed with a surfactant such as hydroxypropylcellulose. Dispersions can also be prepared in glycerol, liquid polyethylene glycols, and mixtures thereof in oils. Illustrative oils are those of petroleum, animal, vegetable, or synthetic origin, for example, peanut oil, soybean oil, or mineral oil. In general, water, saline, aqueous dextrose and related sugar solution, and glycols, such as propylene glycol or polyethylene glycol, are preferred liquid carriers, particularly for injectable solutions. Under ordinary conditions of storage and use, these preparations contain a preservative to prevent the growth of microorganisms.

Pharmaceutical formulations of the inhibitor suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol), suitable mixtures thereof, and vegetable oils.

In addition to the formulations described previously, the inhibitor may also be formulated as a depot preparation. Such long acting formulations may be formulated with suitable polymeric or hydrophobic materials (for example as an emulsion in an acceptable oil) or ion exchange resins, or as sparingly soluble derivatives, for example, as a sparingly soluble salt.

Effective doses of the inhibitor vary depending upon many different factors, including phase of the disease, means of administration, target site, physiological state of the patient, other medications or therapies administered, and physical state of the patient relative to other medical complications. Treatment dosages need to be titrated to optimize safety and efficacy.

As described supra, effective doses of the compositions of the present application vary depending upon many different factors, including type and stage of disease, means of administration, target site, physiological state of the patient, other medications or therapies administered, and physical state of the patient relative to other medical complications. Treatment dosages need to be titrated to optimize safety and efficacy.

Another aspect of the present application relates to a method for inhibiting myofibroblastic and osteogenic differentiation of aortic valvular interstitial cells and/or aortic valvular endothelial cells. The method involves administering, to the aortic valvular interstitial cells and/or aortic valvular endothelial cells, an inhibitor of the nuclear factor kappa B (NFκB) signaling pathway to inhibit myofibroblastic and osteogenic differentiation of the cells.

As described above, the aortic valve is populated by valvular interstitial cells (VICs) and valvular endothelial cells (VECs). In the development of calcific aortic valve disease, inflammation of the aortic valve precedes calcification (Abdelbaky et al., “Early Aortic Valve Inflammation Precedes Calcification: a Longitudinal FDG-PET/CT Study,” Atherosclerosis 238:165-172 (2015), which is hereby incorporated by reference in its entirety). As shown in the Examples described herein, this inflammation, driven by NFκB, can drive reactivation of embryonic-like endothelial to mesenchymal transformation (EndMT) in VECs, which precedes the induction of myofibroblastic and osteogenic differentiation in these cells. In VICs, such inflammation induces myofibroblastic and osteogenic differentiation in VICs also. Thus, as described herein, inhibition of NFκB-mediated inflammation in VECs and VICs can mitigate the myofibroblastic and osteogenic differentiation of these cells, which play a role in the progression of calcific aortic valve disease.

Thus, in accordance with this aspect of the present application, an inhibitor of the nuclear factor NFκB signaling pathway is administered to VECs and/or VICs in order to inhibit myofibroblastic and osteogenic differentiation of the cells.

Inhibitors of the NFκB signaling pathway as well as modes of administration of such inhibitors are described above. In one embodiment, the administering step is carried out in vivo.

In one embodiment, the administration of the NFκB signaling pathway inhibitor inhibits EndMT in valvular endothelial cells. As shown in the Examples described infra, EndMT in the postnatal aortic valve is characterized by coexpression of endothelial (CD31) and mesenchymal (αSMA) phenotypic markers. Therefore, absence of these markers would indicate inhibition of EndMT in the VECs.

In certain embodiments, myofibroblastic and osteogenic differentiation of aortic valvular interstitial cells and/or valvular interstitial cells is inhibited. As shown in the Examples below, phenotypic characterization of VECs and VICs can be examined by evaluating changes in expression for myofibroblastic activation marker αSMA, transcription factor Runx2 required for osteogenic transdifferentiation, and later-stage osteogenic marker OCN. Therefore, absence of these markers would indicate inhibition of myofibroblastic and osteogenic differentiation in these cell populations.

### EXAMPLES

The examples below are intended to exemplify the practice of embodiments of the disclosure but are by no means intended to limit the scope thereof.

### Materials and Methods

Transgenic mouse model. Mouse experiments were performed according to the guidelines of the National Institute of Health and the protocols approved by the Institutional Animal Care and Use Committee of Cornell University and Albert Einstein College of Medicine.

The VEC-specific Nfatc1-enhancer Cre (Nfatc1^{enCre} [nuclear factor of activated T-cells, cytoplasmic 1]) mouse line expresses the Cre enzyme in a subset of valve endocardial populations, which do not give rise to VIC during embryonic valvulogenesis, thus retaining a VEC phenotype (Wu et al., “Nfatc1 Coordinates Valve Endocardial Cell Lineage Development Required for Heart Valve Formation,” Circ Res. 109:183-192 (2011), which is hereby incorporated by reference in its entirety). The Nfatc1-knock-in Cre (Nfatc1^{Cre}) mouse line expresses Cre in valve endocardium which gives rise to both VEC and VIC populations during embryonic valvulogenesis (Wu et al., “Endocardial Cells form The Coronary Arteries by Angiogenesis through Myocardial-Endocardial VEGF Signaling,” Cell 151:1083-1096 (2012), which is hereby incorporated by reference in its entirety)). The Karin lab (University of California, San Diego) kindly provided a mouse with IKKβ-floxed allele (Egan et al., “IkappaB-Kinasebeta-Dependent NF-kappaB Activation Provides Radioprotection to the Intestinal Epithelium,” Proc Natl Acad Sci USA 101:2452-2457 (2004), which is hereby incorporated by reference in its entirety)). Cre-mediated excision of IKKβ would thus prevent canonical NFκB p65/RelA activation.

The mice were crossed with LDLR (low-density lipoprotein receptor)^{-/-} mice from Jackson Labs (No. 2270). Mice were fed a high-fat diet (Harlan Teklad No. TD88137, 42% of calories from fat, 0.25% cholesterol) from 4 to 20 weeks of age, utilized in combination with the homozygous LDLR-knockout background to establish aortic valve disease, as motivated previously (Drolet et al., “A High Fat/High Carbohydrate Diet Induces Aortic Valve Disease in C57BL/6J Mice,” J Am Coll Cardiol 47:850-855 (2006), which is hereby incorporated by reference in its entirety)). For fate-tracking, mice were crossed with Rosa26 reporter mice (Jax No. 003474), expressing floxed-lacZ reporter gene in areas of Cre activity.

All mice were on a C57BL6J substrain background, backcrossed for genetic homogeneity for >5 generations. The resulting animals had genomic deletion of the LDLR, with or without whole valve or VEC-specific inactivation of the IKKβ gene and expression of the lacZ reporter in areas of Cre activity.

These lines were utilized to establish the following mouse groups: Wild-type control (LDLR^{-/-};IKKβ^{+/+};Nfatc1^{+/+} with control diet, N=8), CAVD (LDLR^{-/-}, IKKβ^{+/+};Nfatc1^{+/+} with high-fat diet, N=17), and CAVD+IKKβ^{valveKO} (LDLR^{-/-};IKKβ^{f/f};Nfatc1^{CRE} with high-fat diet, N=9), and CAVD+IKKβ^{vecKO} (LDLR^{-/-};IKKβ^{f/f};Nfatc1^{enCre} with high-fat diet, N=15).

Echocardiography. Atrioventricular function was assessed at 5 months of age using echocardiography according to established procedures. Mice were anesthetized using inhaled 1.8% isoflurane and cradled in the left lateral recumbent position. Abdominal hair was removed using a chemical depilatory cream, and warmed ultrasound transmission gel was applied to the anterior thorax. An ultrahigh-frequency linear array probe (MS550D 22-55 MHz transducer; Vevo) was applied in the long-axis position to the chest. The imaging probe was coupled to the Vevo 2100 system (VisualSonics). Aortic valve leaflet dynamics and ventricular dilation were imaged using the ECG-Gated Kilohertz Visualization software (VisualSonics), which captures 5000 fps to create high-resolution videos of cardiac movement. M-mode was used to measure diameter of the aortic root, just above the aortic sinus. Transvalvular velocity and pressure gradients were captured using the pulsed-wave Doppler mode and manual tracing of >3 velocity time intervals on the resulting plots.

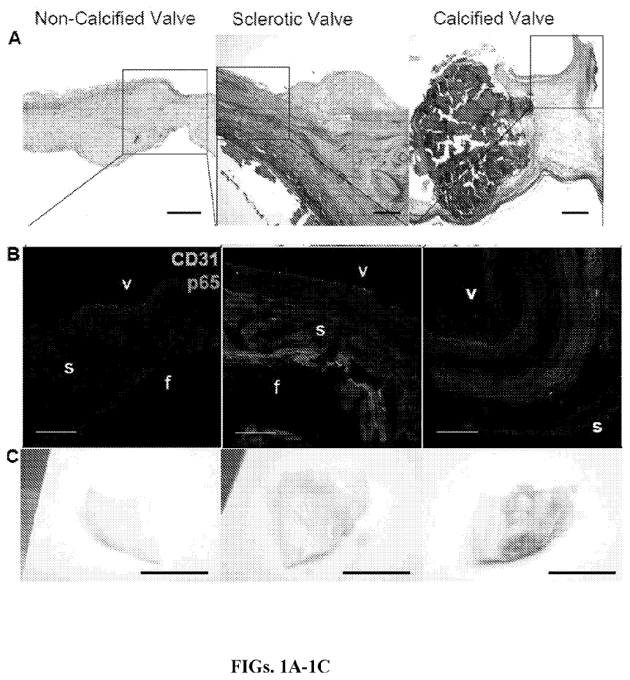

Human Aortic Valves. Noncalcified human aortic valves were obtained from adult (>18 years old) patients undergoing heart transplant for nonvalve related diseases at Children's Hospital in Seattle WA (5 participants total). These valves were free from valvular dysfunction, including endothelial activation commonly present in aged nondiseased heart transplant patients, as previously described (Farrar et al., “Endothelial-derived Oxidative Stress Drives Myofibroblastic Activation and Calcification of the Aortic Valve,” PLoS One 10(4):e0123257 (2015); Holliday et al., “Discovery of Shear- and Side-Specific mRNAs and miRNAs in Human Aortic Valvular Endothelial Cells,” Am J Physiol Heart Circ Physiol. 301:H856-H867 (2011), which are hereby incorporated by reference in their entirety)). Sclerotic and calcified human valves were obtained from patients undergoing valve replacement surgery at Robert Packer Hospital in Sayre, PA. Patient age range was 65 to 90 years, with mean age of 76.2 years (21 participants total). Valves were categorized as either sclerotic or calcified based on macroscopic assessment and histological analysis (e.g., Movat Pentachrome, see FIG. 1A and FIG. 1B). The Institutional Review Board at Seattle Children's Hospital, Guthrie Institutional Review Board at Robert Packer Hospital, and the Institutional Review Board for Human Participants at Cornell University approved all procedures (IRB No. 0908-24, Gene expression and phenotypic changes in stenotic aortic valves). Written informed consent was obtained from all participants.

Histology. Mouse hearts and human aortic valves were isolated, perfused, fixed, embedded in paraffin, sectioned, and mounted. Slides were deparaffinized through xylene and graded ethanol and rinsed in deionized water. For Alizarin Red S (ARS) staining, cells were deparaffinized, hydrated, incubated in ARS stain for 2 minutes, rinsed in xylene and xylene-acetone (1 minute each), dehydrated in 3 changes of xylene (1 minute each), and mounted. Analysis of ARS was done in ImageJ, using thresholding to identify boundaries of the leaflet section. Leaflet section with background subtracted was measured for area and integrated density of ARS stain. Similar deparaffinization and hydration was performed for von Kossa, followed by a 20-minute incubation in 1% aqueous silver nitrate under UV light. Slides were rinsed in water followed by 5% sodium thiosulfate (5 min), rinsed, and counterstained with 0.1% nuclear fast red (5 minutes). Dehydration and mounting performed as above. Slides were imaged and thresholded in ImageJ so that only black-brown mineral deposits were selected for analysis. Mineral deposit area was measured and normalized to valve leaflet area. Russell-Movat pentachrome stain was performed according to manufacturer's instructions, with the following adjustments: 30 seconds in 2% ferric chloride, 20 minutes in 1% alcian blue, and 1 minute in each change of 5% phosphotungstic acid. A color deconvolution algorithm was used to separate individual dyes from the Russell-Movat stained sections into single-channel images. MATLAB was used to find the integrated density of each single-channel image. Integrated density of each component (glycosaminoglycan, collagen, elastin) was normalized to the area of the valve leaflet section being examined. The resulting output was divided by the average integrated density of each dye in control samples, producing fold change expression of glycosaminoglycan, elastin, and collagen relative to control.

Primary Aortic Valve Cell Isolation and Culture. Porcine valves are a widely used analog for human adult valve endothelial and interstitial cells. Unlike mice, pigs develop atherosclerosis and valvular lesions without intervention, similar to humans. Large-scale culture of porcine valve cell populations is feasible due to ample tissue size/cell dissociation yields. Valvular endothelial cells (VEC) and valvular interstitial cells (VIC) were harvested from porcine aortic valves (Shirk Meats, Dundee, NY) following collagenase digestion and screened for phenotype and purity using quantitative real-time polymerase chain reaction (RT-PCR) and immunofluorescence, as demonstrated previously (Gould et al., “Isolation of Valvular Endothelial Cells,” J Vis Exp. 46:pii:2158 (2010), which is hereby incorporated by reference in its entirety)). Only VEC cultures with consistent CD31 and VE-cadherin (vascular endothelial cadherin) expression, cobblestone morphology, and nondetectable αSMA (alphasmooth muscle actin) expression were used. αSMA levels were measured via RT-PCR (>37 cycle threshold), Western blot, and immunofluorescence.

All reagents were obtained from Sigma Aldrich, St. Louis, MO, unless otherwise noted. Porcine aortic valve endothelial cells were cultured in flasks coated with 50 μg/mL rat-tail collagen I (BD Biosciences, San Jose, CA) at 37 C and 5% CO₂ in DMEM supplemented with 10% FBS (Invitrogen, Grand Island, NY), 1% penicillin/streptomycin (Invitrogen, Grand Island, NY), and 50 U/mL heparin sodium salt. Porcine aortic valve interstitial cells were cultured in tissue-culture treated flasks at 37 C° and 5% CO₂ in DMEM supplemented with 10% FBS, 1% penicillin-streptomycin. Cells were used at passage 3 to 5 and 3 to 6 for porcine aortic valve endothelial cells and porcine aortic valve interstitial cells, respectively.

3D Hydrogel Culture Model Preparation. Cellularized type II collagen hydrogels were prepared as previously described (Butcher et al., “Valvular Endothelial Cells Regulate the Phenotype of Interstitial Cells in Co-Culture: Effects of Steady Shear Stress,” Tissue Eng. 12:905-915 (2006), which is hereby incorporated by reference in its entirety)). Briefly, porcine aortic valve endothelial cells were topically seeded at 50,000 cell/cm² density, whereas porcine aortic valve interstitial cells were encapsulated within the neutralized collagen gel suspension (2 mg/mL) at 1,000,000 cells/mL density. Constructs were allowed to gel for 1 hour at 37° C., 5% CO₂, and then cultured overnight in their respective basal-medium conditions. For osteogenic differentiation conditions, basal-medium was supplemented with 10 mmol/L β-glycerophosphate, 50 μg/mL 1-ascorbic acid, and 100 nM dexamethasone, as previously described (Richards et al., “Side-Specific Endothelial-Dependent Regulation of Aortic Valve Calcification: Interplay of Hemodynamics and Nitric Oxide Signaling,” Am J Pathol. 182:1922-1931 (2013), which is hereby incorporated by reference in its entirety). For exogenous TNFα administration, recombinant human TNFα was added to appropriate media condition before administration at 30 ng/mL.

Plasmid Transfection. Cell transfections were conducted using plasmid delivery to VEC or VIC using the 100 μL Neon Electroporation System (Life Technologies) according to the manufacturer's instructions. GFP (green fluorescent protein)-RelA and empty vector were a gift from Warner Greene (Addgene plasmid No. 23255). pBabe-GFP-IKBα-mut (super repressor) and empty vector were a gift from William Hahn (Addgene plasmid No. 15264). Electroporation conditions were 1200 V, 20 ms, 2 pulses. Ten micrograms of plasmid construct per 1,000,000 cells were used. Cells were trypsinized, electroporated with corresponding plasmid construct, and cultured for 24 hours in 5% serum, antibiotic-free DMEM before being seeded into or onto hydrogels.

Hydrogel Calcification Analysis. To quantify calcified matrix developed in different experimental conditions, an Alizarin Red absorbance assay was implemented. Collagen hydrogels were fixed with paraformaldehyde and then incubated with 40 nmol/L ARS dye. This dye binds to calcium crystals in cells or matrix fibers, revealing a red color. Unbound solution was washed out overnight under gentle rocking. Bound ARS dye was then released from the gels using 10% acetic acid, followed by neutralization with 10% ammonium hydroxide. The concentration of dye in solution was then quantified using absorbance spectroscopy at 405-nm wavelength. Samples absorbance readings were normalized to basal growth medium culture condition.

Serum Cholesterol Analysis. Mouse serum cholesterol quantification was performed using the Cholesterol Quantitation Kit as per manufacturer's instructions (Sigma Aldrich, St. Louis, MO). Murine serum was obtained via right ventricle puncture and blood draw at time of mouse euthanasia and isolated using BD vacutainer blood collection tubes (BD, Franklin Lakes, NJ).

Quantitative Real-Time Polymerase Chain Reaction. Total RNA was extracted using a RNeasy total RNA purification kit (Qiagen, Valencia, CA), and RNA was reverse transcribed to cDNA using the iScript cDNA synthesis kit (Bio-Rad, Hercules, CA). Quantitative RT-PCR was performed on all samples using SYBR Green PCR master mix (Applied Biosystems, Foster City, CA) and a CFX96 or MiniOpticon Real-Time PCR Detection System (Bio-Rad, Hercules, CA). Samples amplifying at >37 cycles were considered nondetectable. Primer sequences are shown in Table 1.

TABLE 1
Primers for Quantitative RT-PCR

| Gene | Forward Primer | Reverse Primer | Accession Number |
|---|---|---|---|
| 18S | TAGAGGGACAAGTGG | AATGGGGTTC | NR_046261.1 |
| | CGT (SEQ ID NO: 1) | AACGGGTT (SEQ ID NO: | |
| | | 2) | |
| ACTA2 | CAGCCAGGATGTGTGA | TCACCCCCTGATGTCTAGG | NM_001164650.1 |
| (αSMA) | AGAA (SEQ ID NO: 3) | A (SEQ ID NO: 4) | |
| Osteocalcin | CTCCAGCCACAACATC | TGGCCTCCAGCACTGTTTA | NM_001164004.1 |
| | CTTT (SEQ ID NO: 5) | T (SEQ ID NO: 6) | |
| PECAM1 | ATCTGCATCTCGTGGG | GAGCTGAAGTGTCAGCAG | NM_213907.1 |
| | AAGT (SEQ ID NO: 7) | GA (SEQ ID NO: 8) | |
| RUNX2 | GCACTACCCAGCCACC | TATGGAGTGCTGCTGGTC | XM_005666074.3 |
| | TTTA (SEQ ID NO: 9) | TG (SEQ ID NO: 10) | |
| SNA1 (snail | GCCCAACTACAGCGAG | CCAGGAGAGAGTCCCAGA | XM_021077961.1 |
| family | CTAC (SEQ ID NO: 11) | TG (SEQ ID NO: 12) | |
| transcriptional | | | |
| repressor 1) | | | |
| CDH5 (VE- | CGTGGTGGAAACACAA | TGTGTACCTGGTCTGGGT | NM_001001649.2 |
| cadherin) | GATG (SEQ ID NO: 13) | GA (SEQ ID NO: 14) | |

Immunofluorescences Staining and Analysis. Human aortic valves and whole mouse hearts were fixed in 4% paraformaldehyde overnight at 4° C. and processed for paraffin sectioning. Slides were deparaffinized through xylene and graded ethanol and rinsed in deionized water. Heat mediated antigen retrieval using citrate buffer, pH 6.0 (Electron Microscopy Sciences, Hatfield, PA), was performed using a commercial pressure cooker for 2 minutes. Tissue sections were subsequently permeabilized with 0.2% Triton-X (VWR International, West Chester, PA).

For in vitro cultures, intact collagen gels were rinsed twice in PBS and fixed in 4% paraformaldehyde for 1 hour at 25° C. Samples were rinsed 3× on a rocker with PBS for 15 minutes each, permeabilized with 0.2% Triton-X (VWR International, West Chester, PA) for 10 minutes on rocker, and blocked in 10% goat serum for 1 hour at 25° C.

The following antibodies were used at the provided dilution concentrations/ratios:

TABLE 2
Human Immunofluorescence (immunohistochemistry-
Paraffin Sections)

| | Manufacturer/ | Concentration/ |
|---|---|---|
| Antibody | Catalog No. | Dilution Ratio |
| Mouse anti- | Cell Signaling | 1:200 |
| human CD31 | Technology: 3528 | |
| Rabbit anti- | Cell Signaling | 1:400 |
| human NFκB p65 | Technology: 8242 | |

TABLE 3
Mouse Immunfluorescence (Immunohistochemistry-
Paraffin Sections)

| | Manufacturer/ | Concentration/ |
|---|---|---|
| Antibody | Catalog No. | Dilution Ratio |
| Mouse anti-human αSMA | Abcam: ab7817 | 10 μg/mL (1:100) |
| Rabbit anti-mouse VCAM1 | Cell Signaling | 1:100 |
| (vascular cell adhesion | Technology: 32653 | |
| molecule 1) | | |
| Rabbit anti-human NFκB p65 | Cell Signaling | 1:400 |
| | Technology: 8242 | |
| Rabbit anti-human ki67 | Abcam: ab15580 | 10 μg/mL (1:100) |
| Rabbit anti-mouse CD31 | Abcam: ab28364 | 1:50 |
| Rabbit anti-human IKKβ | Abcam: ab124957 | 10 μg/mL (1:100) |
| (inhibitor of nuclear | | |
| factor kappa-B | | |
| kinase subunit beta) | | |

TABLE 4
Porcine Immunofluorescence (Whole-Mount IF)

| | | Concentration/ |
|---|---|---|
| Antibody | Catalog No. | Dilution Ratio |
| Mouse anti-human αSMA | Abcam: ab7817 | 10 μg/mL (1:100) |
| Rabbit anti-human VE- | Cell Signaling | 1:200 |
| Cadherin | Technology: 2500 | |
| Mouse anti-human | Abcam: ab76956 | 10 μg/mL (1:100) |
| Runx2 (runt-related | | |
| transcription factor 2) | | |
| Mouse-anti human Osteocalcin | Abcam: ab13420 | 10 μg/mL (1:100) |
| Rabbit anti-human NFκB p65 | Cell Signaling | 1:100 |
| | Technology: 8242 | |

Samples were incubated overnight at 4° C., rinsed 3× in PBS on rocker for 15 minutes each, and species-specific secondary antibodies raised in goat conjugated to Alexa Fluor 488 or 568 fluorophores were added at 4 μg/mL (1:500) dilution ratio (Invitrogen, Grand Island, NY).

Samples were incubated for 2 hours at room temperature, rinsed, then incubated for an additional 30 minutes with Draq5 nuclear stain (Enzo Life Sciences AG, Lausen, Switzerland, 1:1000). Samples were rinsed thoroughly in PBS 3× on rocker for 15 minutes each, then imaged using a Zeiss 710 (Thornwood, NY) laser scanning confocal microscope.

TUNEL (terminal deoxynucleotidyl transferase dUTP nickend labeling) assay (Invitrogen, Grand Island, NY) was performed according to manufacturer's instructions to label apoptosis, necrosis, and cell nuclei. Cell proliferation was assessed using ki67 immunostaining (1:100 rabbit anti-human; Abcam). Tissue sections were imaged with a Zeiss 710 laser scanning confocal microscope, analyzed, and quantified using ImageJ. Number of cell nuclei and cells positive for apoptosis/proliferation per section were counted using particle analysis. Percent of cells positive for apoptosis (TUNEL stain) or proliferation (Ki67) were presented.

Nuclear-integrated density analysis was performed using ImageJ. Slides were imaged and thresholded using IgG isotype controls so that only positive protein-expression regions were selected for analysis. Images were segmented using DAPI-stained nuclear-positive regions to create a region of interest for analysis; MATLAB was used to find the integrated density for protein expression within the nuclear region of interest map. Nuclear-integrated density was then normalized to cell count within the field of view.

Statistical Analysis. Unless stated otherwise, 1-way ANOVA in conjunction with Tukey honestly significant difference post hoc test for parametric data sets; nonparametric analysis was conducted using Kruskal-Wallis test. Experimental groups were compared to one another with 1-way ANOVA in conjunction with Tukey honestly significant difference post hoc test; adjusted P<0.05 were considered statistically significant, data are presented as mean±SEM, unless stated otherwise.

### Example 1—NFκB Activity Increases in a Layer Specific Manner in Proportion to CAVD Severity in Human Aortic Valves

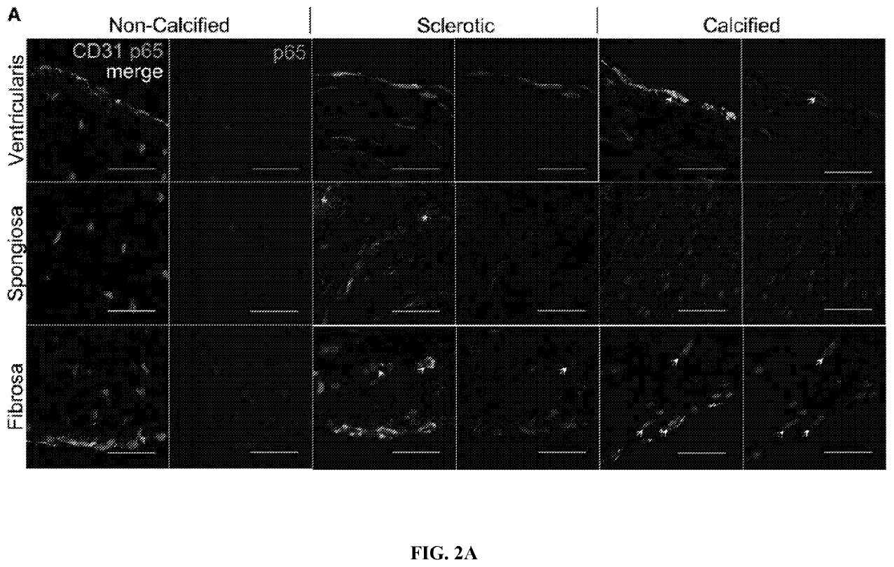

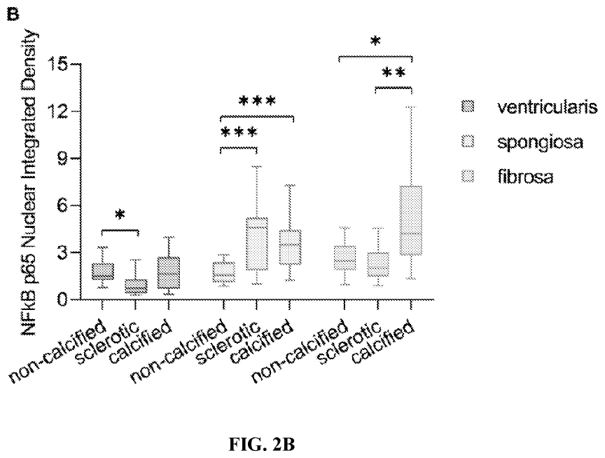

First, changes in NFκB activity in relation to calcific aortic valve pathology and human disease progression were quantified. Noncalcified human aortic valve leaflets were thin, exhibited intact and proper tri-layer morphology, and contiguous endothelialization (FIG. 1A). Diseased human valves, in contrast, were thickened, exhibited disrupted matrix architecture, and collagenglycosaminoglycan rich or fibrotic-calcified nodule presentation (FIG. 1A and FIG. 1C). Sclerotic valve leaflets were associated with greater collagen-glycosaminoglycan rich lesions, whereas calcified valves had greater fibrotic/calcified lesions with greater matrix disruption (FIG. 1A). Corresponding sections were contained for CD31 and NFκB p65 to indicate inflammatory signaling activity in relation to the VEC spatial localization (FIG. 2A). In noncalcified valves, nuclear p65 signal was found to be elevated within the fibrosa region, whereas cellular expression within the ventricularis and fibrosa was not statistically different, as assessed by normalized nuclear-colocalized protein signal integrated density (FIG. 2B). In sclerotic valves, cells of the ventricularis region demonstrated lower p65 expression compared to that of the spongiosa or fibrosa. p65 expression was highly colocalized with CD31, consistent with the endothelium undergoing inflammatory dysfunction (FIG. 2A). In some of the sclerotic valve sections, signs of neovessel formation were observed to occur within the intimal space (FIG. 2A, asterisks), as well as CD31-positive cells independent of neo-vessel formation (FIG. 2A, yellow arrows). Assessment of p65 nuclear-integrated density indicated the ventricularis of sclerotic valves as having the lowest nuclear-colocalized 65 signal as compared to either the spongiosa or fibrosa regions (FIG. 2B). Within calcified valves, however, this p65 presentation and nuclear localization were more pronounced within the subendothelial, interstitial populations (FIG. 2A, arrows). Nuclear p65 expression was greater in calcified valves compared to the sclerotic valves regardless of valvular regions (FIG. 2B). These results determine that p65 expression and nuclear colocalization in aortic valves increases with calcific disease severity. Furthermore, nuclear active p65 is fibrosa side-specific but develops significant ventricularis involvement during late-stage CAVD.

### Example 2—Valve-Specific IKKβ Inactivation Prevents p65/RelA NFκB Signal Transduction without Perturbing Postnatal Aortic Valve Structure or Function

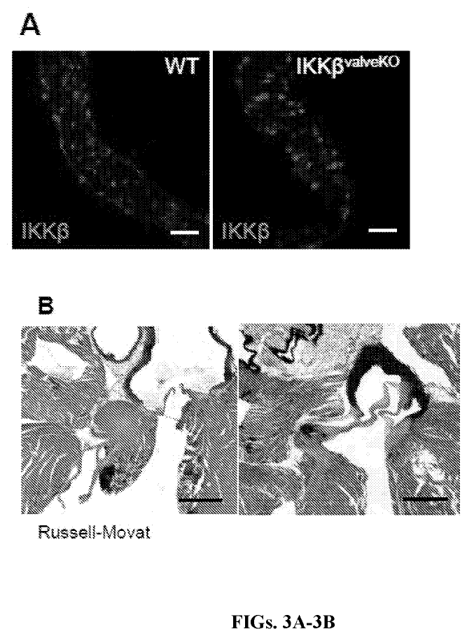

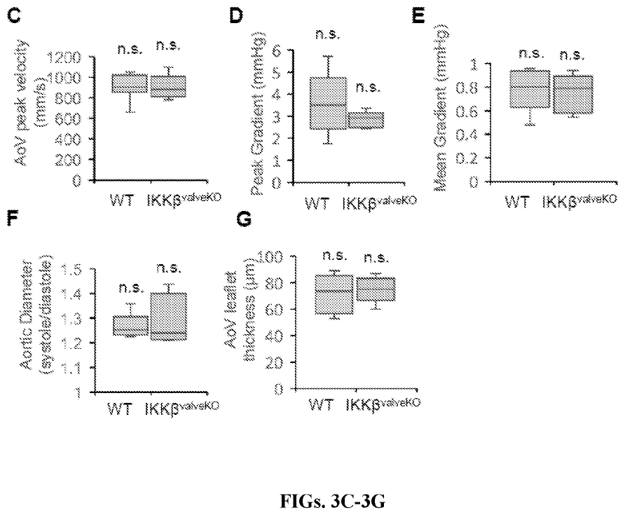

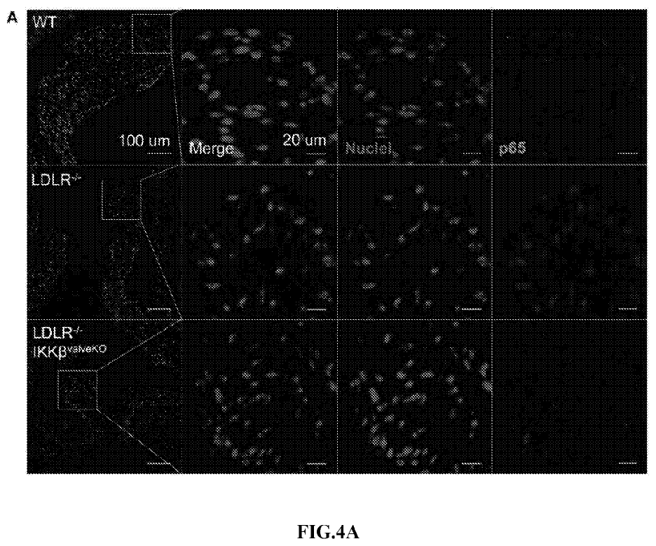

To ascertain the role of NFκB signaling contributing to aortic valve homeostasis or CAVD pathogenesis, NFκB was first inactivated by the deletion of IKKβ in VEC and VIC using the valve-specific NFATc1-enhancer Cre (Nfatc1^{Cre}) mouse (Wu et al., “Endocardial Cells form The Coronary Arteries by Angiogenesis through Myocardial-Endocardial VEGF Signaling,” Cell 151:1083-1096 (2012), which is hereby incorporated by reference in its entirety)). This mouse was further crossed with the established hypercholesterolemia-mediated CAVD risk model background (LDLr^{-/-}), which then progressively develops CAVD when fed a Western diet (Drolet et al., “A High Fat/High Carbohydrate Diet Induces Aortic Valve Disease in C57BL/6J Mice,” J Am Coll Cardiol 47:850-855 (2006), which is hereby incorporated by reference in its entirety)). It was determined that valve-specific deletion of IKKβ (FIG. 3A) after embryonic valvulogenesis did not affect heart morphogenesis in age-matched adult mice without a CAVD background (FIG. 3B). Aortic valve peak velocity, peak transvalvular gradient, and mean transvalvular gradient were not affected by IKKβ deletion (IKK^{valveKO} thereafter; FIG. 3C, 3D, 3E). Neither aortic root diameter nor valve leaflet thickness significantly differed from their wild-type counterparts (FIGS. 3F and 3G). Cytosolic p65 was expressed primarily in the endothelium with minimal nuclear activation (FIG. 4A).

However, nuclear localization of p65 was significantly increased with mice that develop CAVD (LDLr^{-/-} with western diet starting at postnatal 4-weeks, thereafter), most prominently in the VEC populations (FIG. 4A) consistent with human CAVD. Valvular (VEC and VIC)-specific deletion of IKKβ in the same CAVD background significantly reduced levels of p65 expression and nuclear activity across both VEC and VIC in the age-matched CAVD+IKKβ^{valveKO} mice. Inactivation of downstream p65 transcriptional activity was confirmed via immunofluorescence for VCAM1, a direct transcriptional target of p65 (FIG. 4B) (Tak et al., “NF-kappaB: A Key Role in Inflammatory Diseases,” J Clin Invest. 107:7-11 (2001), which is hereby incorporated by reference in its entirety)). VCAM1 was expressed prominently in endothelium of CAVD mice, but not in CAVD+IKKβ^{valveKO}. As the NFκB signaling cascade is an important mechanism of cell survival (Luo et al., “IKK/NF-kappaB Signaling: Balancing Life and Death-A New Approach to Cancer Therapy,” J Clin Invest. 115:2625-2632 (2005), which is hereby incorporated by reference in its entirety)), it was determined whether its deletion would adversely affect either cell apoptosis (FIG. 4C) or proliferation (FIG. 4D). As expected, both of these parameters were upregulated in CAVD conditions. However, in CAVD+IKKβ^{valveKO}, neither cell apoptosis nor proliferation were statistically different from their wild-type controls (FIGS. 4E and 4F). These results demonstrate that valve-specific lack of NFκB signaling is dispensable for murine aortic valve morphogenesis and homeostasis, but NFκB is activated during adult murine CAVD.

### Example 3—Valve-Specific Inactivation of IKKβ Prevents Hypercholesterolemia-Mediated Aortic Valve Pathogenesis

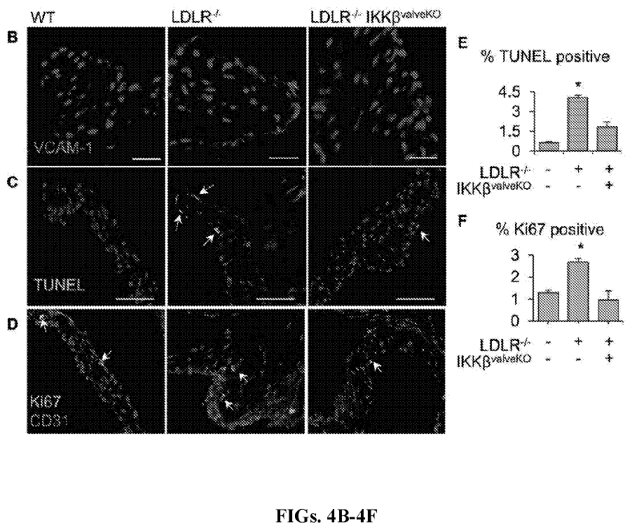

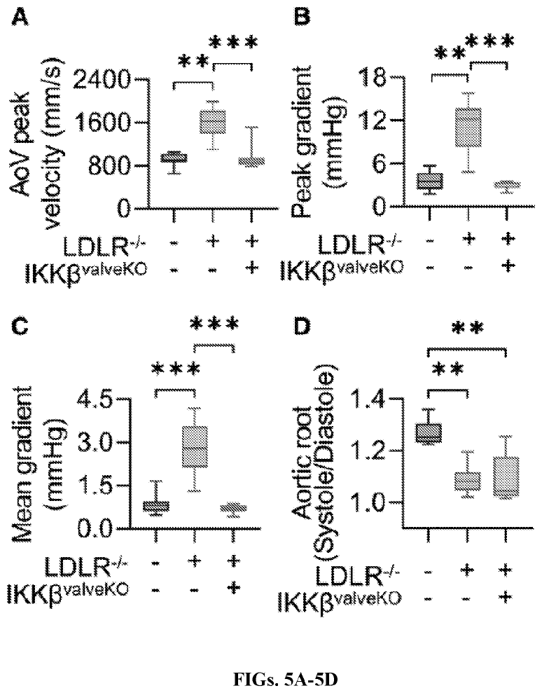

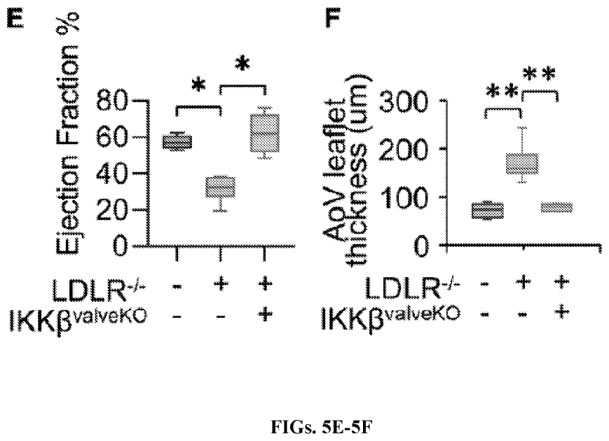

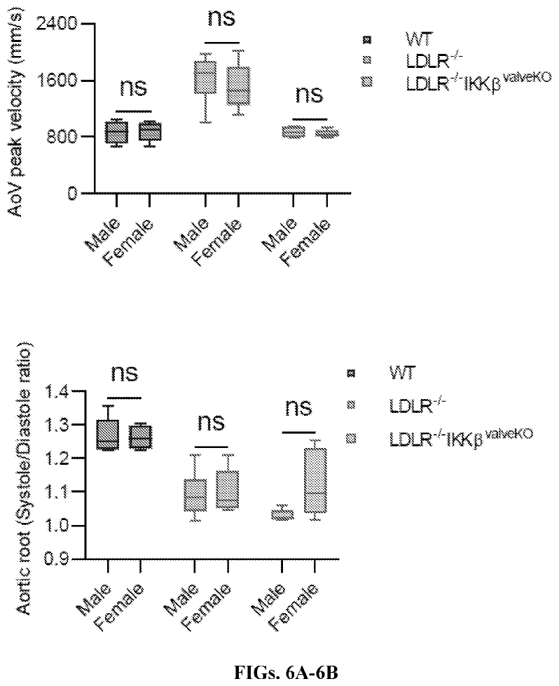

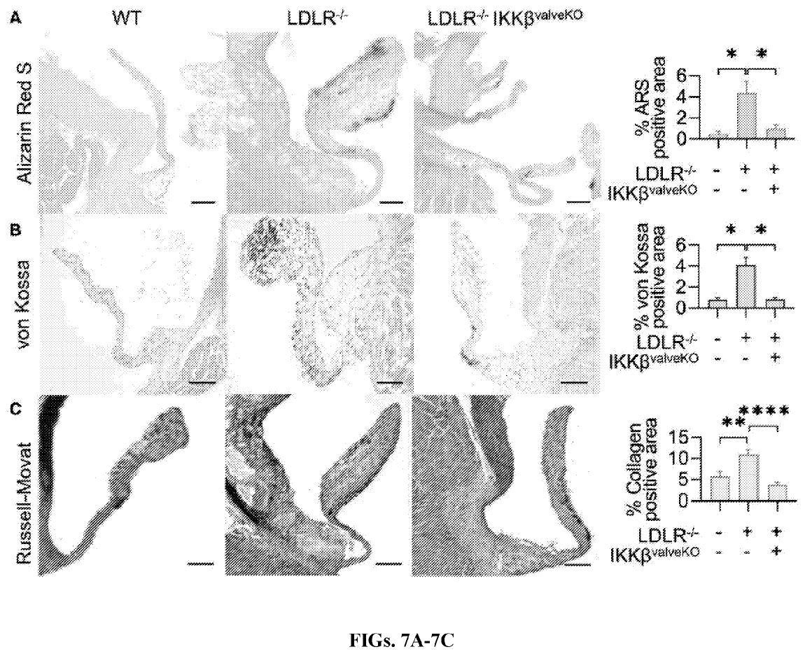

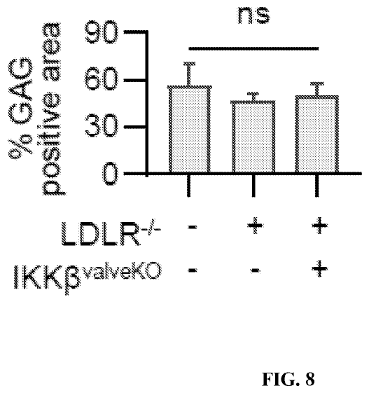

It was next determined how valve-specific NFκB inactivation modulates hypercholesterolemia-induced aortic valve degeneration and hemodynamic stenosis. Echocardiographic analysis revealed increased aortic valve peak velocity, peak gradient, and mean gradient (FIG. 5A, 5B, SC) in CAVD mice in comparison to wild-type controls, but not in CAVD+IKKβ^{valveKO}. Measurement of the aortic root during peak systole and peak diastole (FIG. 5D) revealed a loss of aortic distensibility in CAVD that was not prevented by CAVD+IKKβ^{valveKO} (FIG. 5D). CAVD+IKKβ^{valveKO} did, however, prevent decreased ejection fraction percentage, which occurred in CAVD mice compared with wild-type counterparts (FIG. 5E). Aortic leaflet thickness measurements indicated that CAVD mice developed significantly thickened leaflets in comparison to either wild-type controls or CAVD+IKKβ^{valveKO} (FIG. 5F). Examination of possible sex-differences in CAVD development of hemodynamic dysfunction or its prevention via valve-specific IKKβ deletion did not indicate statistical differences between strain matched males or females (FIGS. 6A-6B). Histological analysis showed increased calcium and mineral deposition as indicated via ARS and von Kossa staining (FIGS. 7A and 7B) and collagen content as indicated via changes in Russell-Movat pigmentation (FIG. 7C) in CAVD mice, but not CAVD+IKKβ^{valveKO}. A nonsignificant trend of reduction in glycosaminoglycan content was observed with both CAVD and CAVD+IKKβ^{valveKO} conditions compared to control (FIG. 8). These results support that valve-specific NFκB inhibition protects against CAVD within a persistent pro-CAVD environment.

### Example 4—p65/RelA Activation Induces VEC Participation in Osteogenic Differentiation and Calcification In Vitro

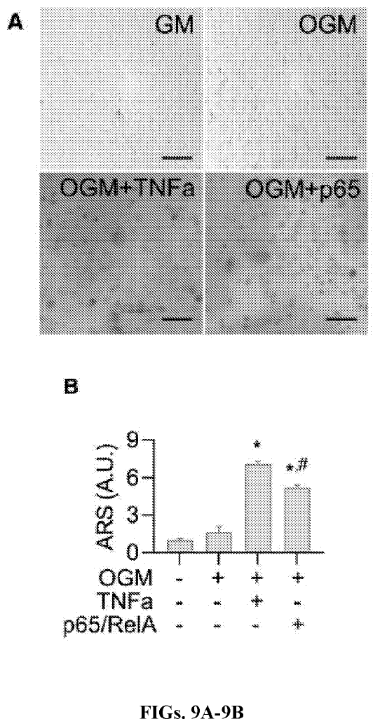

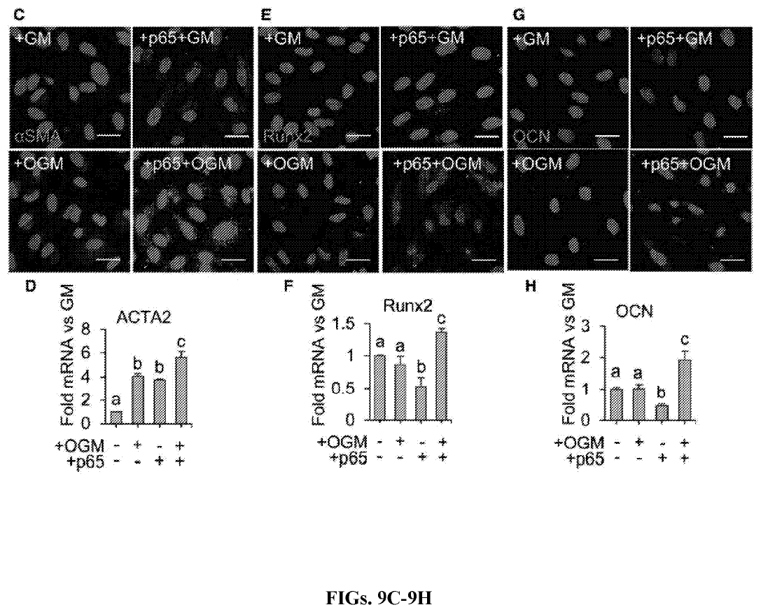

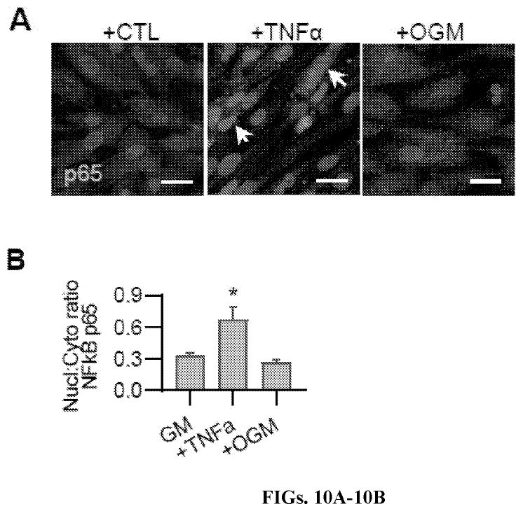

Considering the protective effect of p65 inhibition in VEC and VIC in vivo, it was next determined how NFκB signaling modulates VEC phenotype in vitro. Porcine aortic VEC were cultured on 3D collagen hydrogels under osteogenic differentiation medium (OGM) conditions. Treatment of VEC for 14 days with OGM was unable to induce calcific deposition indicated by ARS staining (FIGS. 9A and 9B). However, cotreatment of OGM with either exogenous TNFα (OGM+TNFα) or forced expression of p65 (OGM+p65) resulted in formation of distinct calcified nodules and significantly increased calcium deposition (FIGS. 9A and 9B). Immunofluorescence staining indicated administration of NFκB adduct such as exogenous TNFα is required to induce p65 nuclear localization, where OGM alone is insufficient (FIGS. 10A-10B). Phenotypic characterization was conducted via quantitative RT-PCR and immunofluorescence staining, evaluating changes in expression for myofibroblastic activation marker αSMA, transcription factor Runx2 (runt-related transcription factor 2) required for osteogenic transdifferentiation, and later-stage osteogenic marker OCN (osteocalcin). OGM treatment and p65 overexpression both significantly increased αSMA gene and protein expression, with the combinatorial administration upregulating it even further (FIGS. 9C and 9D). Neither OGM or p65 overexpression alone significantly affected Runx2 gene or protein expression (FIGS. 9E and 9F). However, the coadministration of +OGM+p65 significantly increased Runx2 gene expression (FIG. 9E), with both cytosolic and nuclear protein expression observed (FIG. 9E). Similar trends were observed with OCN (FIGS. 9G and 9H). Neither OGM nor p65 overexpression resulted in observable protein expression of OCN (FIG. 9G). OGM did not significantly alter OCN gene expression, whereas p65 overexpression resulted in a significant decrease in OCN gene expression (FIG. 9H). However, the coadministration of +OGM+p65 resulted in cytosolic OCN protein expression and significant increase in OCN gene expression (FIGS. 9G and 9H). These results establish that chronic inflammatory mediated NFκB activation is required to induce myofibroblastic and osteogenic differentiation of VEC in vitro.

### Example 5—p65/RelA NFκB Signaling is Active in Pro-Osteogenic VIC Differentiation, and its Inhibition Mitigates Differentiation and Calcification In Vitro

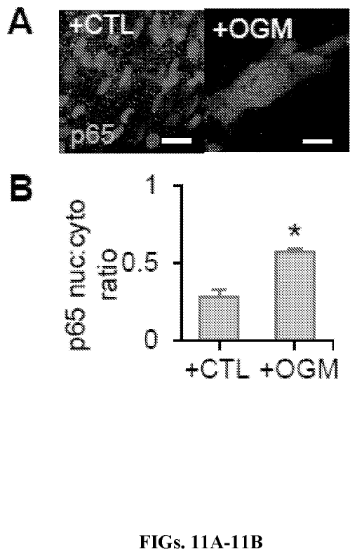

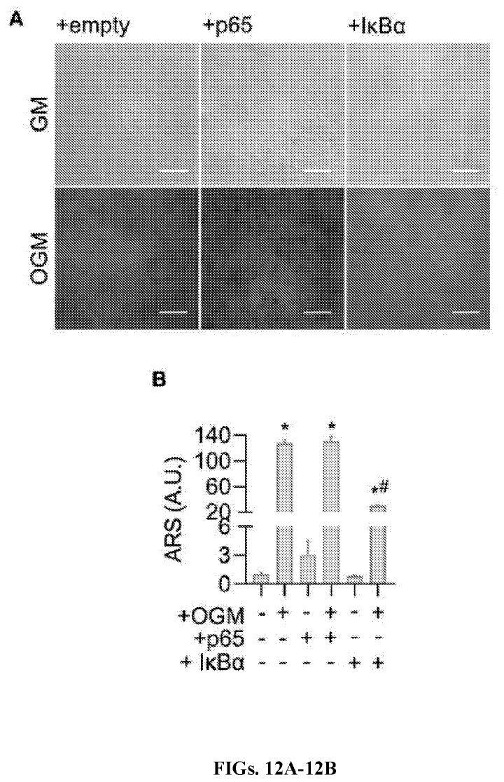

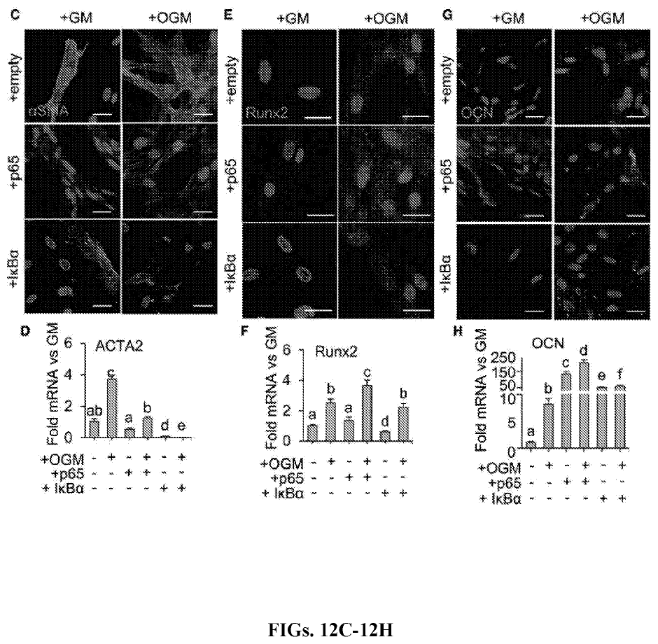

It was next determined how p65 activity regulates VIC calcification. Porcine VIC were transfected with empty, p65, or IκBα mutant repressor vectors, encapsulated within 3D collagen hydrogels, and cultured for 14 days with or without OGM treatment. It was found that OGM alone induced a significant increase in VIC p65 nuclear expression after 14 days of culture (FIGS. 11A and 111B). Overexpression of p65 exacerbated VIC calcification response to OGM, and inhibition of p65 (+IκBa super repressor) mitigated VIC calcification response as indicated by ARS staining and quantification (FIGS. 12A and 12B). Alterations to cell phenotype were assessed via quantitative RT-PCR and immunostaining for gene and protein expression. αSMA gene expression was upregulated due to OGM culture, with both control and OGM cells displaying protein expression (FIGS. 12C and 12D). OGM cultured VIC displayed significant increases to both Runx2 and OCN gene expression (FIGS. 12F and 12H) and observable Runx2 and OCN protein expression compared with controls (FIGS. 12E and 12G). Surprisingly, overexpressing p65 resulted in OCN gene expression undergoing an over 100-fold increase compared with control, with observable protein expression (FIGS. 12G and 12H), despite the lack of significant changes to αSMA and Runx2 gene or protein expression (FIGS. 12C and 12F). The combination of OGM and p65 overexpression (+OGM+p65) exacerbated Runx2 and OCN gene and protein expression, significantly increasing both as compared to OGM or p65 expression alone (FIG. 12E-12H). αSMA gene expression for +OGM+p65 VIC was not significantly different from control (FIG. 12D). Phenotypic assessment for the p65 loss of function (via IκBα overexpression), indicated a reduction of both αSMA and Runx2 expression compared with control (FIG. 12E-12H). Inhibition of p65 did not mitigate changes in OCN protein expression, despite an over 50-fold increase to gene expression (FIGS. 12G and 12H). Inhibition of p65 mitigated increases in αSMA in OGM culture (+IκBα+OGM; FIG. 12A). Coadministration (+IκBα+OGM) yielded an intermediary phenotype of VIC in either condition alone (+OGM or +IκBα). Runx2 was elevated and comparable to OGM alone (FIGS. 12E and 12F), whereas gene expression increases for OCN were comparable to IκBa overexpressing VIC (FIG. 12H). These results show that NFκB activity exacerbates OGM induced VIC myofibroblastic and osteogenic differentiation in vitro, and its inhibition significantly but not completely reduces this response.

### Example 6—p65 Mediates Inflammatory EndMT in VEC In Vitro and In Vivo

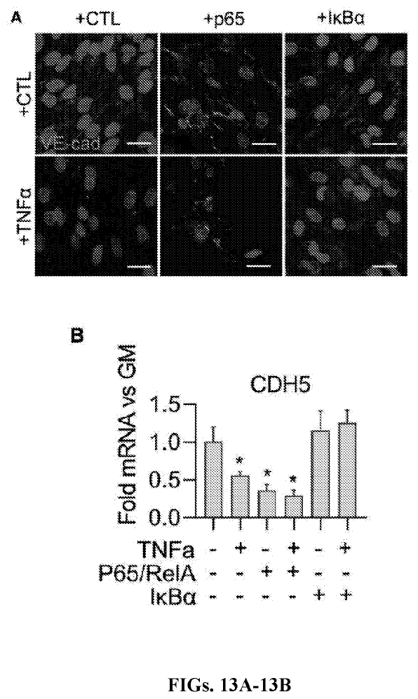

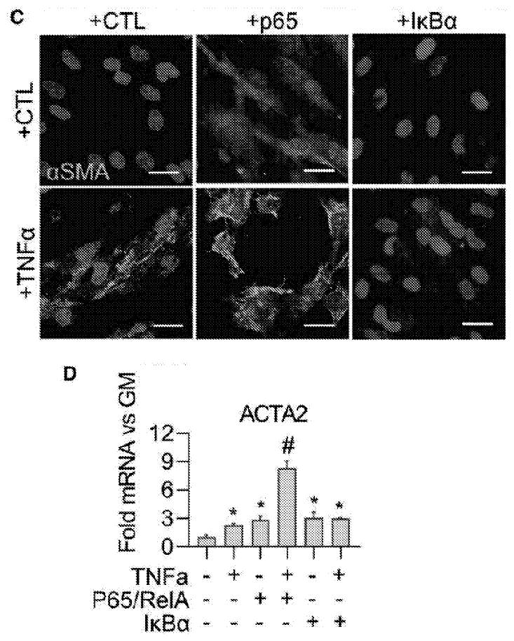

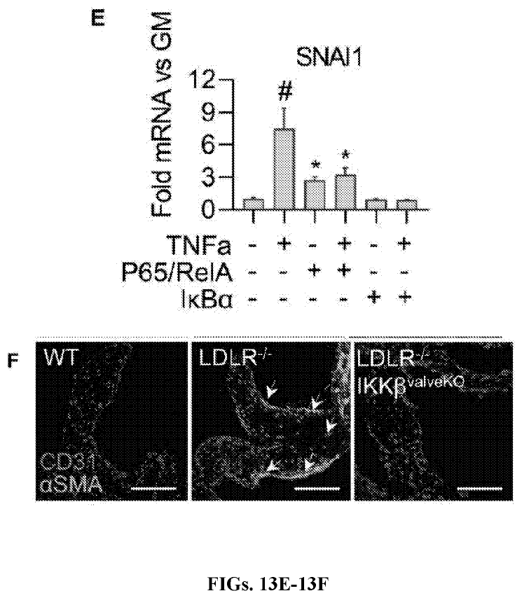

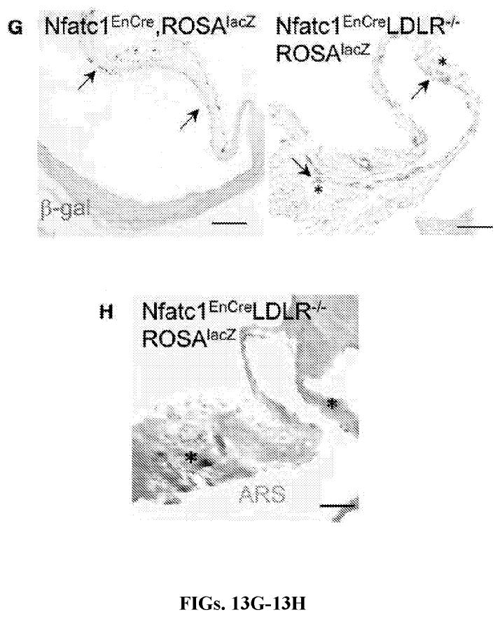

The previous data identified reduced susceptibility of VEC to osteogenic media compared with VIC. Therefore, it was next tested whether inflammatory activation of NFκB mediates VEC transformation into a phenotypically distinct plastic state, via endothelial-to-mesenchymaltransition (EndMT). Potential phenotypic transdifferentiation via loss or disruption VE-cadherin concomitant with increased expression of myofibroblastic marker αSMA and increased expression of transcription factor SNAI1 (snail family transcriptional repressor 1), crucial for dissolution of E-cadherin junctions during EndMT, was evaluated. Both exogenous TNFα and p65 overexpression induced EndMT in that period, characterized by disruption of VE-cadherin junctions and reduction in gene expression (CDH5; FIGS. 13A and 13B), concomitant with increased αSMA (FIGS. 13AC and 13D). SNAI1 gene expression was also significantly increased exogenous TNFα and p65 overexpression (FIG. 13E), canonical p65 inhibition, via the overexpression of IκBα, prevented EndMT induction (FIG. 13A-13E). These results support that the induction of osteogenic activity in VEC requires a prior transition to a mesenchymal phenotype via EndMT in vitro.

EndMT in postnatal aortic valve in vivo is characterized by coexpression of endothelial (CD31) and mesenchymal ((SMA) phenotypic markers (Mahler et al., “Inflammatory Cytokines Pro-Mote Mesenchymal Transformation in Embryonic and Adult Valve Endothelial Cells,” Arterioscler Thromb Vasc Biol. 33:121-130 (2013), which is hereby incorporated by reference in its entirety) but its direct demonstration requires lineage tracking. In the wild-type background, little to no expression of αSMA was observed and a robust, intact CD31-positive endothelial layer (FIG. 13F, left). In CAVD mice, αSMA expression was elevated, most prominently within cells coexpressing CD31 and αSMA (FIG. 13F, middle, arrows). To further confirm the valve endothelial origins of these EndMT-VEC populations, versus potential circulating progenitor or extravasation of recruited inflammatory populations, the Nfatc1^{enCre} was backcrossed with the LDlr^{-/-}, along with a Rosa26lacZ reporter line. β-galactosidase (β-gal) was restricted to the valve endocardial lining observed in non-CAVD mice (FIG. 13G, left), demonstrating that EndMT does not occur during aortic valve homeostasis. However, with CAVD induction via chronic high-fat diet in the Ldlr^{-/-} background, β-galactosidase staining identified substantial disruption of the β-gal+ endothelial lining on the fibrosa surface, with cells that submigrated into the tissue interstitium and colocalize with calcified lesions (FIGS. 13G and 13H, black arrows and asterisks). Furthermore, CAVD+IKKβ^{eKO} mice had αSMA expression comparable to wild-type controls and no observable cells with coexpression of CD31 and αSMA (FIG. 13F, right). These results directly demonstrate the involvement of EndMT in CAVD in vivo, but not during homeostasis, and NFκB inactivation in valves prevents VEC EndMT in vivo.

### Example 7—VEC-Specific Inactivation of IKKβ Inhibits Hypercholesterolemia-Mediated Aortic Valve Calcification

To further clarify the role of VEC-specific NFκB activity in CAVD degeneration, a VEC-specific deletion of IKKβ in the CAVD background (Nfatc1^{enCre};LDLr^{-/-};IKKβ^{fl/fl} supplemented with western diet, henceforth LDLR^{-/-}IKKβ^{vecKO}) was performed.

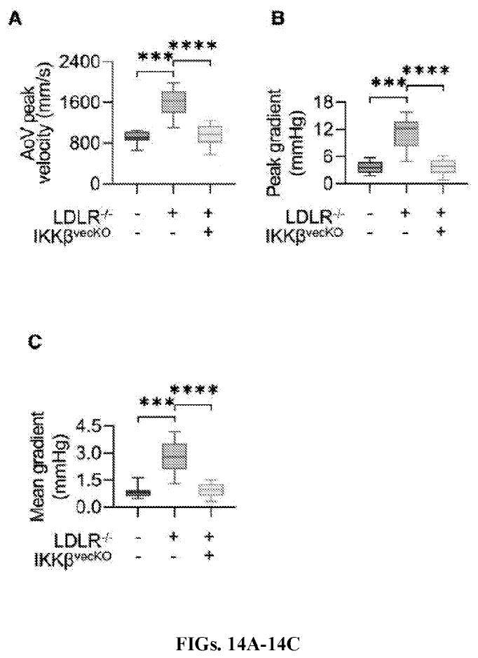

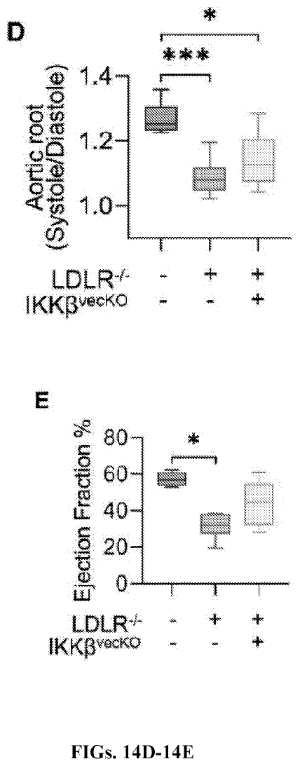

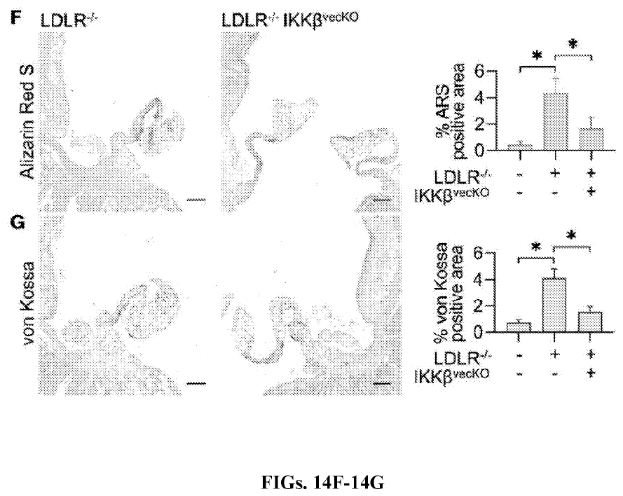

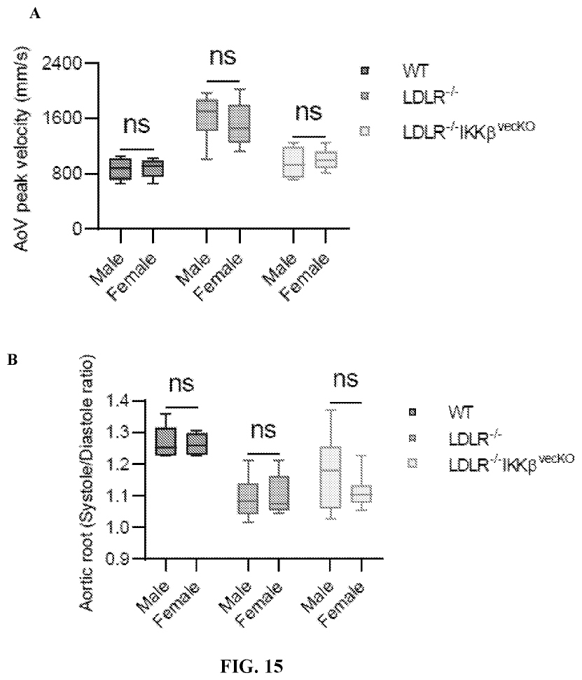

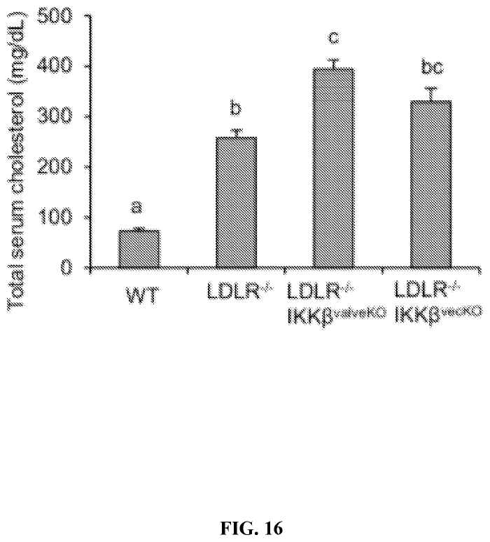

Echocardiographic analysis of the LDLR^{-/-}IKKβ^{vecKO} mice showed significant improvement of valvular hemodynamic function. Aortic valve jet velocity, peak gradient, and mean gradient were improved in the Nfatc1^{enCre} (VEC-specific) versus the CAVD background (FIG. 14A, 14B, 14C). Aortic root distensibility and ejection fraction percentage for the VEC-specific IKKβ deletion were not statistically different from the CAVD background (FIGS. 14D and 14E). As with the whole-valve deletion strategy, the male and female populations were parsed to examine possible sex-specific differences in CAVD induced hemodynamic dysfunction but significant differences between sexes were not found (FIG. 15). Histological assessment of valvular calcium and mineral deposition via ARS and von Kossa staining, respectively, demonstrated that VEC-specific IKKβ deletion was able to achieve a statistically significant reduction of calcium/mineral deposition compared with CAVD background, similarly with the whole-valve-specific deletion (FIGS. 14F and 14G). Analysis of total serum cholesterol indicated both the whole-valve and VEC-specific IKKβ deletion maintained a hypercholesterolemic state (FIG. 16). Taken together, these results demonstrate that VEC-specific inhibition ameliorates valvular pathogenesis without affecting associated coronary heart disease.

### Discussion of Examples 1-7

The results of this study establish the importance of the p65/RelA NFκB pathway in CAVD pathogenesis and its potential as a target for pharmacological treatment. Inactivation of canonical NFκB pathway via valvular deletion of IKKβ prevented valve endothelial inflammation in vivo in CAVD risk environments. p65 control of pathogenic valvular transitions was conserved in vitro and in vivo, across VEC and VIC, and whether osteogenic medium or hypercholesterolemic diet induced. Inflammation is a hallmark of both early and late stages of CAVD (Cote et al., Inflammation is Associated with the Remodeling of Calcific Aortic Valve Disease,” Inflammation 36:573-581 (2013); New et al., “Molecular Imaging Insights into Early Inflammatory Stages of Arterial and Aortic Valve Calcification,” Circ Res. 108:1381-1391 (2011), which are hereby incorporated by reference in their entirety)) with important repercussions for both endothelial (Farrar et al., “Endothelial-derived Oxidative Stress Drives Myofibroblastic Activation and Calcification of the Aortic Valve,” PLoS One 10(4):e0123257 (2015); Mahler et al., “Inflammatory Cytokines Pro-Mote Mesenchymal Transformation in Embryonic and Adult Valve Endothelial Cells,” Arterioscler Thromb Vasc Biol. 33:121-130 (2013), which are hereby incorporated by reference in their entirety)) and interstitial (Yu et al., “Tumor Necrosis Factor-Alpha Accelerates the Calcification of Human Aortic Valve Interstitial Cells Obtained from Patients with Calcific Aortic Valve Stenosis Via the BMP2-Dlx5,” J Pharmacol Exp Ther. 337:16-23 (2011), which is hereby incorporated by reference in its entirety)) cell participation in disease. The observed mitigation of inflammation (VCAM1 expression) was not accompanied by lowered serum cholesterol; therefore, the downstream improvements in valve function here cannot be attributed to the reduction of general cardiovascular risk factors, in contrast to prior studies that ultimately were not translatable to humans (Miller et al., “Lowering Plasma Cholesterol Levels Halts Progres-Sion of Aortic Valve Disease in Mice,” Circulation. 119:2693-2701 (2009), which is hereby incorporated by reference in its entirety)). Furthermore, specifically targeting VEC IKKβ activity was sufficient in retaining normative valvular function despite a persistent pro-CAVD environment. It should be noted that although IKKβ activity is important to mediate canonical NFκB signal transduction, its pleiotropic activities extend to regulation of processes including nuclear β-catenin and Runx2 signaling (Lamberti et al., “Regulation of Beta-Catenin Function by the IkappaB Kinases,” J Biol Chem. 276:42276-42286 (2001); Al-Huseini et al., “Deletion of IkB-Kinase β in Smooth Muscle Cells Induces Vascular Calcification through O-Catenin-Runt-Related Transcription Factor 2 Signaling,” J Am Heart Assoc. 7:1-15 (2018); Schröfelbauer et al., “NEMO Ensures Sig-Naling Specificity of the Pleiotropic IKKβ by Directing its Kinase Activity Toward IκBα,” Mol Cell. 47:111-121 (2012), which are hereby incorporated by reference in their entirety)). IKKα and IKKβ regulate transcriptional β-catenin signaling, with it being demonstrated that TNF upregulates β-catenin in the vasculature with atherogenesis (Al-Aly et al., “Aortic Msx2-Wnt Calcification Cascade is Regulated by TNF-alpha-Dependent Signals in Diabetic Ldlr−/− Mice,” Arterioscler Thromb Vasc Biol. 27:2589-2596 (2007), which is hereby incorporated by reference in its entirety)). Nevertheless, the combination of in vitro primary cell culture models coupled with in vivo IKKβ conditional deletion models point to the key role of the IKKβ-p65/RelA relay in VEC and VIC as relevant to CAVD. This demonstrated mechanism between the reduction of inflammatory activation and amelioration of endothelial dysfunction resulting in improved valve function emphasizes the opportunity for early valve endothelial-specific diagnostic markers and treatment strategies but also the utility of in vivo CAVD models for testing valve-specific pathogenic mechanisms and their targeting.

The present application demonstrates a role for p65 as a gatekeeper for VEC initiation and participation in valvular calcific progression by connecting inflammatory activation to a downstream osteogenic phenotype. Activation of p65 may control a sequential phenotypic shift from quiescent to osteogenic progenitors via EndMT, as supported by studies showing TNFα promotes osteogenic differentiation of mesenchymal stem cells via IKKβ activation (Hess et al., “TNFalpha Promotes Osteogenic Differentiation of Human Mesenchymal Stem Cells by Triggering the NF-kappaB Signaling Pathway,” Bone 45:367-376 (2009), which is hereby incorporated by reference in its entirety)). EndMT has been implicated in coronary arterial pathobiology (Chen et al., “Endothelial-to-Mesenchymal Transition Drives Atherosclerosis Progression,” J Clin Invest. 125:4514-4528 (2015); Evrard et al., “Endothelial to Mesenchymal Transition is Common in Atherosclerotic Lesions and is Associated with Plaque Instability,” Nat Commun. 7:11853 (2016), which are hereby incorporated by reference in their entirety)), and presence of myofibroblast populations are elevated throughout both early and late stages of valvular calcification (Latif et al., “Expression of Smooth Muscle Cell Markers and Co-Activators in Calcified Aortic Valves,” Eur Heart J 36:1335-1345 (2015), which is hereby incorporated by reference in its entirety)). The results here provide first direct in vivo evidence for NFκB activity driving postnatal EndMT within the aortic valve. This phenomenon could share mechanisms with oncology, as NFκB is known to be essential for endothelial-to-mesenchymal transition in breast cancer (Huber et al., “NF-kappaB is Essential for Epithelial-Mesenchymal Transition and Metastasis in a Model of Breast Cancer Progression,” J Clin Invest. 114:569-581 (2004), which is hereby incorporated by reference in its entirety)). In postnatal epithelial cells, p65 and Snail1 coregulate endothelial to-mesenchymal transition, possibly through p65 nuclear recruitment of Snail1 (Stanisavljevic et al., “The p65 Subunit of NF-κB and PARP1 Assist Snail1 in Activating Fibronectin Transcription,” J Cell Sci. 124(pt 24):4161-4171 (2011), which is hereby incorporated by reference in its entirety)). EndMT of postnatal VEC has been demonstrated in multiple in vitro contexts, including aberrant mechanical stimulation (Balachandran et al., “Cyclic Strain Induces Dual-Mode Endothelial-Mesenchymal Transformation of the Cardiac Valve,” Proc Natl Acad Sci USA. 108:19943-19948 (2011), which is hereby incorporated by reference in its entirety)), consequence of inflammation (Mahler et al., “Inflammatory Cytokines Promote Mesenchymal Transformation in Embryonic and Adult Valve Endothelial Cells,” Arterioscler Thromb Vasc Biol. 33:121-130 (2013), which is hereby incorporated by reference in its entirety)), and in response to TGFβ (transforming growth factor β). Similarities between postnatal and developmental EndMT suggests that EndMT may provide a source of interstitial progenitor populations with increased differentiation potential. Recent studies have shown that aortic valves contain a subpopulation of cells displaying phenotypic plasticity, collectively referred to as mesenchymal progenitors; VIC displaying differentiation potential into osteogenic, adipogenic, chondrogenic, and myofibroblast lineages (Chen et al., “Identification and Characterization of Aortic Valve Mesenchymal Progenitor Cells with Robust Osteogenic Calcification Potential,” Am J Pathol. 174:1109-1119 (2009), which is hereby incorporated by reference in its entirety)). One source is circulating endothelial progenitor cells, but these account for only 4% of cells with demonstrated phenotypic plasticity in sclerotic valves (Skowasch et al., “Cells of Primarily Extra-Valvular Origin in Degenerative Aortic Valves and Bioprostheses,” Eur Heart J. 26:2576-2580 (2005), which is hereby incorporated by reference in its entirety)). Postnatal VEC undergoing EndMT in healthy valves were unable to be identified via genetic lineage tracing, suggesting that VEC EndMT does not participate in postnatal valve homeostasis. In contrast, the findings demonstrate that postnatal VEC in CAVD risk conditions are uniquely reenabled to undergo EndMT by p65/RelA activation as an initiating component of CAVD.

Among valvular interstitial populations, aortic-derived VIC uniquely display an enhanced inflammatory agonist response, expressing higher levels of proinflammatory and pro-osteogenic mediators (Yang et al., “Pro-Osteogenic Phenotype of Human Aortic Valve Interstitial Cells is Associated with Higher Levels of Toll-like Receptors 2 and 4 and Enhanced Expression of Bone Morphogenetic Protein 2,” J Am Coll Cardiol. 53:491-500 (2009), which is hereby incorporated by reference in its entirety)). It is demonstrated that p65 enhances VIC calcific turnover, and inhibition of p65 signaling mitigates that participation. This mechanism may underlie the finding that tissue stiffness induces VIC activation and calcification (Yip et al., “Calcification by Valve Interstitial Cells is Regulated by the Stiffness of the Extracellular Matrix,” Arterioscler Thromb Vasc Biol. 29:936-942 (2009), which is hereby incorporated by reference in its entirety)) as tissue stiffness is known to activate NFκB in certain cancer cell types mediated by actomyosin contractions (Ishihara et al., “Substrate Stiffness Regulates Temporary NF-κB Activation Via Actomyosin Contractions,” Exp Cell Res. 319:2916-2927 (2013), which is hereby incorporated by reference in its entirety)). NFκB could be a mechanotransductive signaling bridge between ECM (extracellular matrix) and calcification in VIC, participating in a positive feedback loop via RhoA-p65 interaction, as demonstrated in renal cells (Wu et al., “TNF Induces Caspase-Dependent Inflammation in Renal Endothelial Cells through a Rho- and Myosin Light Chain Kinase-Dependent Mechanism,” Am J Physiol Renal Physiol. 297:F316-F326 (2009), which is hereby incorporated by reference in its entirety)). The mitigating effects of the VEC-specific NFκB inhibition on total valve pathology are possibly due to retained homeostatic function, as the role of VEC in maintaining VIC quiescence and promoting valvular health has been demonstrated (Richards et al., “Side-Specific Endothelial-Dependent Regulation of Aortic Valve Calcification: Interplay of Hemodynamics and Nitric Oxide Signaling,” Am J Pathol. 182:1922-1931 (2013); El Accaoui et al., “Aortic Valve Sclerosis in Mice Deficient in Endothelial Nitric Oxide Synthase,” Am J Physiol Heart Circ Physiol. 306:H1302-H1313 (2014), which are hereby incorporated by reference in their entirety)). VEC actively regulate the structural and mechanical properties of the aortic valve (Aikawa et al., “Human Semilunar Cardiac Valve Remodeling by Activated Cells from Fetus to Adult: Implications for Postnatal Adaptation, Pathology, and Tissue Engineering,” Circulation 113:1344-1352 (2006); El-Hamamsy et al., “Endothelium-Dependent Regulation of the Mechanical Properties of Aortic Valve Cusps,” J Am Coll Cardiol. 53:1448-1455 (2009), which are hereby incorporated by reference in their entirety), which leads to downstream changes in both VIC and VEC signaling and disease propagation. Collectively, the findings indicate that diet-induced hypercholesterolemic CAVD initially presents with p65 activity prominently within the VEC, which radiates further into the tissue interstitium in later-stage disease. Although this study primarily focused on the role of the tissue-resident endothelial and interstitial populations, it is also important to note that other cellular constituents may have a role in systemic inflammatory processes underpinning CAVD progression, such as leukocytes and T cells. Examination of murine and human tissue sections indicate concomitant increase of infiltrating monocyte and macrophage populations with valvular matrix dysregulation/valvular degeneration (Hulin et al., “Macrophage Transitions in Heart Valve Development and Myxomatous Valve Disease,” Arterioscler Thromb Vasc Biol. 38:636-644 (2018), which is hereby incorporated by reference in its entirety)). In IL-1Ra (interleukin-1 receptor antagonist) deficient mice, which develop onset of aortic valve stenosis, it has been indicated that T cells were thought to be the primary mediators, with pathogenesis being reversed by combined deletion of TNF (Isoda et al., “Deficiency of Interleukin-1 Receptor Antagonist Induces Aortic Valve Disease in BALB/c Mice,” Arterioscler Thromb Vasc Biol. 30:708-715 (2010), which is hereby incorporated by reference in its entirety)). The results further support the requirement for exogenous cardiovascular inflammation triggering conditions, such as chronic hypercholesterolemia as probed within this body of work and supported in other studies (Nus et al., “Diet-Induced Aortic Valve Disease in Mice Haploinsufficient for the Notch Pathway Effector RBPJK/CSL,” Arterioscler Thromb Vasc Biol. 31:1580-1588 (2011), which is hereby incorporated by reference in its entirety)) to initiate misregulated valvular cell activity mediated via canonical NFκB signaling. From the in vitro results, it was further observed that the combination of both p65 activation and osteogenic stimulus is necessary for VEC calcific activity. This suggests that VEC require exogenous triggering mechanisms, such as underlying coronary arterial inflammation, to induce an EndMT mediated transdifferentiation to a phenotypically plastic intermediary.

Familial hypercholesterolemia, a common hereditary disease in the Western hemisphere, is a disorder afflicting lipoprotein metabolism giving rise to chronic systemic inflammation and to increase risk to atherosclerotic development and coronary heart disease (Cuchel et al., “European Atherosclerosis Society Consensus Panel on Familial Hyper-Cholesterolaemia. Homozygous Familial hypercholesterolaemia: New Insights and Guidance for Clinicians to Improve Detection and Clinical Management. A position Paper from the Consensus Panel on Familial Hypercholesterolae-Mia of the European Atherosclerosis Society,” Eur Heart J. 35:2146-2157 (2014), which is hereby incorporated by reference in its entirety)). Less appreciated, however, is the increased incidence of CAVD in familial hypercholesterolemia patients, with over half of males and 21% to 41% females with homozygous familial hypercholesterolemia exhibiting aortic regurgitation or advanced valvular dysfunction (Kawaguchi et al., “Characteristic Cardiovascular Manifestation in Homozygous and Heterozygous Familial Hypercholesterolemia,” Am Heart J. 137:410-418 (1999); Rajamannan et al., “Hypercholesterol-Emic Aortic-Valve Disease,” N Engl J Med 349:717-718 (2003), which are hereby incorporated by reference in their entirety)). Given the relative insufficiency of lipid-lowering or antiatherogenic therapeutics in addressing the valvular degeneration (Cowell et al., “Scottish Aortic Stenosis and Lipid Lowering Trial, Impact on Regression (SALTIRE) Investigators. A Randomized Trial of Intensive Lipid-Lowering Therapy in Calcific Aortic Stenosis,” N Engl J Med. 352:2389-2397 (2005); Rossebo et al., “SEAS Investigators. Intensive Lipid Lowering with Simvastatin and Ezetimibe in Aortic Steno-Sis,” N Engl J Med. 359:1343-1356 (2008); Antonini-Canterin et al., “Stage-Related Effect of Statin Treatment on the Pro-Gression of Aortic Valve Sclerosis and Stenosis,” Am J Cardiol. 102:738-742 (2008), which are hereby incorporated by reference in their entirety)), a closer examination of valvular cell behavior in the context of hypercholesterolemia-induced coronary heart disease is warranted. Furthermore, there is greater appreciation within the field of possible sex-differences in valvular calcific progression (Porras et al., “Calcific Aortic Valve Disease: A Battle of the Sexes,”. Circ Res. 120:604-606 (2017); Masjedi et al., “Sex-Related Differences in Matrix Remodeling and Early Osteogenic Markers in Aortic Valvular Interstitial Cells,” Heart Vessels. 32:217-228 (2017), which are hereby incorporated by reference in their entirety)). However, between the hypercholesterolemia-induced CAVD model or corresponding valvular conditional deletion models, sex-specific differences in the disease cause were not denoted, possibly due to the early time point of assessment; such differences may become more apparent at later stages of stenosis/calcific progression. It is shown here that inhibition of NFκB signaling is sufficient to benefit valve physiological functionality while underlying hypercholesterolemia and coronary/arterial disease remains. This suggests that NFκB dependent signaling mechanisms downstream of elevated serum cholesterol and subsequent coronary inflammation may be the primary mediator of valvular pathogenesis.

The results of this study present NFκB p65 as a novel therapeutic target for CAVD. The findings indicate that both VEC and VIC calcific transition and pathogenic activity converge at canonical NFκB mediated signaling, regardless of initiating context. It was found that modulating this signaling axis in both VEC and VIC populations, versus VEC alone, to be more effective in preventing CAVD development. This is hypothesized to be due to VIC having its own VEC-independent program of activation and osteogenic transition. The dynamics of these interactions and concomitant activity should be studied further. Supporting this approach of NFκB targeting are recent findings that osteoprotegerin mitigates CAVD calcification (Weiss et al., “Osteoprotegerin Inhibits Aortic Valve Calcification and Preserves Valve Function in Hypercholesterolemic Mice,” PLoS One. 8:e65201 (2013), which is hereby incorporated by reference in its entirety)). Osteoprotegerin is a decoy receptor for RANKL (receptor activator of nuclear factor-KB ligand) (Wright et al., “RANK, RANKL and Osteoprotegerin in Bone Biology and Disease,” Curr Rev Musculoskelet Med. 2:56-64 (2009), which is hereby incorporated by reference in its entirety)) which activates the canonical NFκB pathway (Hoffmann et al., “Circuitry of Nuclear Factor kappaB Signaling,” Immunol Rev. 210:171-186 (2006), which is hereby incorporated by reference in its entirety)). RANKL is necessary for bone formation and is upregulated in calcified aortic valves (Kaden et al., “Receptor Activator of Nuclear Factor kappaB Ligand and Osteoprotegerin Regulate Aortic Valve Calcification,” J Mol Cell Cardiol. 36:57-66 (2004), which is hereby incorporated by reference in its entirety)), supporting NFκB signaling modulation as the potential mechanism behind the efficacy of osteoprotegerin treatment. Although a role for NFκB in governing valve EndMT and calcification has been demonstrated, it has yet to be shown whether pharmacological targeting can mitigate CAVD progression without eliciting adverse systemic effects or toxicity. Furthermore, although the genetic strategy provides some indication NFκB targeting as a potential prophylactic strategy, it must be denoted that Cre-mediated genomic recombination and therefore IKK silencing occurs during embryonic valvular development. It remains to be seen whether in the more clinically relevant context of adult disease onset, these NFκB targeting strategies will be sufficient to ameliorate CAVD progression. In the context of acute cardiovascular injury, pharmacological agents directly targeting NFκB canonical signaling activity, such as pyrrolidine-dithiocarbamate and BAY 11-7082, have been shown to be effective in treatment of septic shock (Hulin et al., “Macrophage Transitions in Heart Valve Development and Myxomatous Valve Disease,” Arterioscler Thromb Vasc Biol. 38:636-644 (2018), which is hereby incorporated by reference in its entirety) or ischemic reperfusion injury (Liu et al., “Inhibition of NF-kappaB Activation by Pyrrolidine Dithiocarbamate Prevents in Vivo Expression of Proinflammatory Genes,” Circulation. 100:1330-1337 (1999); Kim et al., “Bay11-7082, A Nuclear Factor-κB Inhibitor, Reduces Inflammation and Apoptosis in a Rat Cardiac Ischemia-Reperfusion Injury Model,” Int Heart J. 51:348-353 (2010), which are hereby incorporated by reference in their entirety)) and, therefore, may be warranted for evaluation in the context of CAVD. NFκB has been the focus of many studies regarding inflammation in cancer development, resulting in many attempts to modulate its function in vivo (Lin et al., “The NF-κB Activation Pathways, Emerging Molecular Targets for Cancer Prevention and Therapy,” Expert Opin Ther Pat. 14:45-55 (2011), which is hereby incorporated by reference in its entirety)). Cross-talk between oncology and CAVD may lead to future avenues for CAVD treatment via NFκB modulation and may be able to capitalize on the novel NFκB mechanisms of CAVD shown here.

### Materials and Methods for Example 8

Animals. Mouse experiments were performed according to the guidelines of the National Institute of Health and the protocols approved by the Institutional Animal Care and Use Committee of Cornell University. The mice used were LDLR (low-density lipoprotein receptor)−/− mice from Jackson Labs (No. 2270). Mice were fed a high-fat diet (Harlan Teklad No. TD88137, 42% of calories from fat, 0.25% cholesterol). Age-matched C57BL6J mice were obtained from Jackson Laboratories (Bar Harbour, ME) for control study.

Drug administration. The NFκB pathway was intervened by administering either BAY11-7082 (injected intraperitoneal 3 times per week at 5 mg/kg) or tetrahydrobiopterin (BH4) (oral gavage at 10 mg/kg/day) to LDLR/mice, for 8 weeks after giving them high fat diet for 20 weeks

Echocardiography. Transthoracic echocardiography was performed under 2% isofluorane anaesthesia, with MS400D and MS5S50D probes connected to a Vevo 2100 system (VisualSonics). Heart valve function was assessed at 6 months and 8 months of age using according to established procedures. Mice were anesthetized using inhaled 2% isoflurane. Abdominal hair was removed using a chemical depilatory cream, and warmed ultrasound transmission gel was applied to the anterior thorax. Probes were applied in the long-axis and short-axis position to the chest. Transvalvular velocity and pressure gradients were captured using the pulsed-wave Doppler mode and manual tracing of >3 velocity time intervals on the resulting plots.

Q-PCR analysis. Mouse Aortic valve samples were homogenized, total RNA was isolated using Takarabio RNA, purified and further quantified via Nanodrop. DNAse I (Invitrogen) treatment was performed, and RNA was reverse transcribed to cDNA using the iScript cDNA synthesis kit (Bio-Rad, Hercules, CA). Quantitative RT-PCR was performed on all samples using SYBR Green PCR master mix (Applied Biosystems, Foster City, CA) and a CFX96 or MiniOpticon Real-Time PCR Detection System (Bio-Rad, Hercules, CA). Samples were amplifying at >37 cycles.

Tissue staining and immunohistochemistry. Mouse hearts were isolated, perfused, fixed, embedded in paraffin, sectioned, and mounted.

Alizarin red staining (ARS). Sections were deparaffinized and incubated in ARS stain for 5 minutes (1% Alizarin red in water, pH 4.2 adjusted by using 10% ammonium hydroxide in water). Sections were incubated in acetone for 30 seconds and then rinsed with a 1:1 solution of acetone and xylene. Finally, sections were rinsed in xylene and mounted in cytoseal. Pictures were acquired using Olympas microscope.

VonKossa staining. Similar deparaffinization and hydration was performed for vonKossa, followed by a 20-minute incubation in 1% aqueous silver nitrate under UV light. Slides were rinsed in water followed by 5% sodium thiosulfate (5 minutes), rinsed, and counterstained with 0.1% nuclear fast red (5 minutes). Finally, sections were rinsed in xylene and mounted in cytoseal. Pictures were acquired using Olympas microscope.

### Example 8—Administration of BAY11-7082 or BH4 in a CAVD Mouse Model

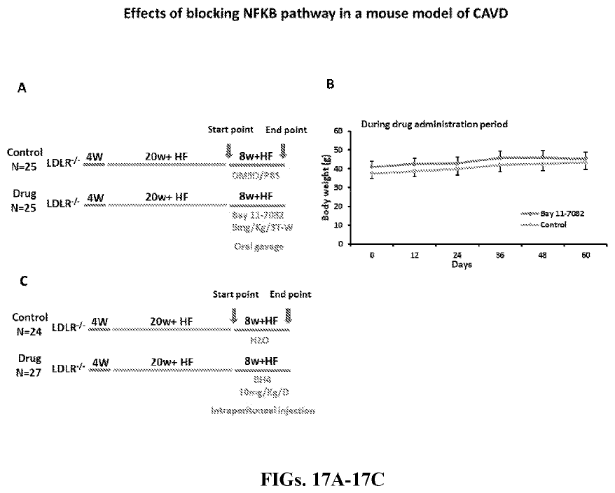

In this work, the NFkB pathway was intervened by administering either BAY11-7082 (injected intraperitoneal 3 times per week at 5 mg/kg) or tetrahydrobiopterin (BH4) (oral gavage at 10 mg/kg/day) to LDLR^{-/-} mice, a CAVD mouse model, for another 8 weeks after starting them on high fat diet for 20 weeks (FIGS. 17A-17C). Cardiac hemodynamic parameters, such as aortic valve peak velocity and left ventricle ejection fraction, were measured using doppler ultrasound before and after BAYT 1-7082 and BH4 administration. At the end of the treatment, mice were sacrificed and whole hearts were extracted for histological staining and aortic valves were used for fibro-osteogenic gene profiling. BAY11-7082 is a potential anti-inflammatory agent that irreversibly inhibits IKKα and thus arrests IkB phosphorylation. BH4 is a co-factor and key regulator of endothelial nitric oxide synthase (eNOS).

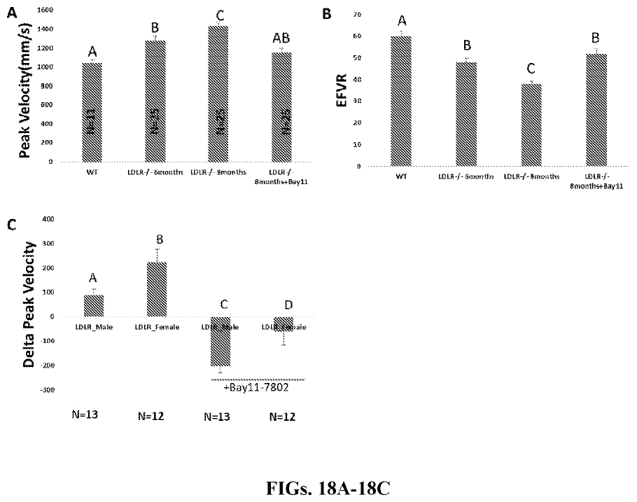

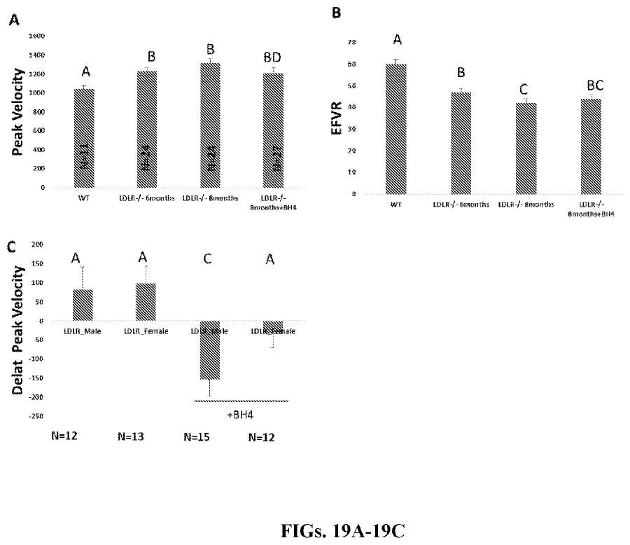

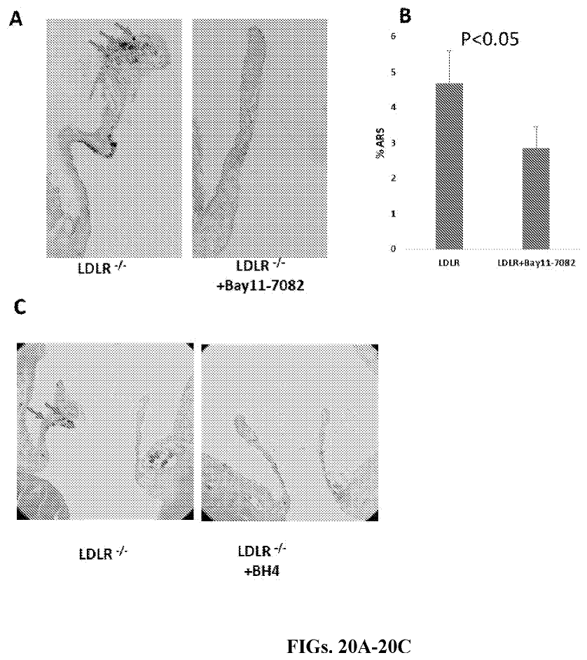

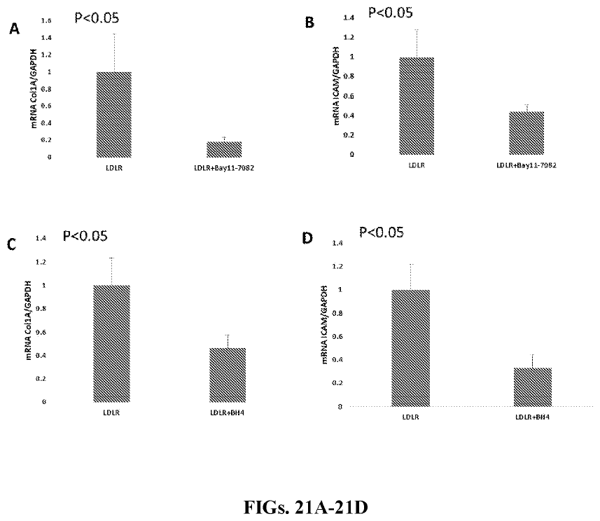

As a result, control LDLR^{-/-} mice that were administered 10% DMSO/PBS had continuous increase in the peak velocity, from 6 months to 8 months, compared to the WT mice (FIG. 18A). The ejection fraction velocity ratio (EFVR) was decreased in control LDLR^{-/-} mice 6 months to 8 months (FIG. 18B). LDLR^{-/-} mice that were given BAY11-7082 starting from 6 months had lessened increase in peak velocity and decrease in EFVR at 8 months (FIGS. 18A and 18B). BAY11-7082 significantly affected both female and male mice (FIG. 18C) while BH4 affected male mice more profoundly than female mice (FIG. 19). Alizarin red staining for calcium contents on the aortic valves showed that there was less calcium deposition on the valves of LDLR^{-/-} mice treated with either BAY11-7082 or BH4 (FIG. 20A-20C). Fibro-osteogenic gene profiling on aortic valve tissues showed that collagen type 1A (Col1A) and intercellular adhesion molecule 1 (ICAM1) were significantly decreased with treatment of either BAY11-7082 or BH4 (FIG. 21A-21D). Taken all together, these results suggest that inhibition to NFkB pathway by either interfering with eNOS or IkB phosphorylation arrest the progression of CAVD by slowing down hemodynamic changes, calcium deposition, and reducing fibro-calcific gene expressions.

To date, there has not yet been any pharmacological treatment to treat or slow down the progression of CAVD. Clinical studies failed to show beneficial effects of existing pharmacological agents towards the course of CAVD (Cowell et al., “Scottish Aortic Stenosis and Lipid Lowering Trial, Impact on Regression (SALTIRE) Investigators. A Randomized Trial of Intensive Lipid-lowering Therapy in Calcific Aortic Stenosis,” N Engl J Med 352:2389-2397 (2005); Rossebo et al., “Intensive Lipid Lowering with Simvastatin and Ezetimibe in Aortic Stenosis,” N Engl J Med 359:1343-1356 (2008); Chan et al., “ASTRONOMER Investigators. Effect of Lipid Lowering with Rosuvastatin on Progression of Aortic Stenosis: Results of the Aortic Stenosis Progression Observation: Measuring Effects of Rosuvastatin (ASTRONOMER) Trial,” Circulation 121:306-314 (2010), which are hereby incorporated by reference in their entirety)).

### Materials and Methods for Example 9

In vitro cell culture. Porcine aortic valve cells were obtained from primary tissue isolates, and maintained in sterile tissue culture conditions (37° C. at 5% CO₂). Cells were seeded at a surface density of 10,000 cells per cm², with medium changes every 48 hours for the duration of the experiment up to 14 days. Cells were subjected to either basal growth medium (Dulbecco's modified medium supplemented with 10% v/v fetal bovine serum, 1% v/v Penicillin-Streptomyocin), or osteogenic differentiation medium which consists of basal growth medium further supplemented with 10 mM β-glycerophosphate, 50 μg/ml L-Ascorbic acid, and 100 nM Dexamethasone.

Drug administration. For pharmacologic studies using the corresponding NFkB inhibitors (i.e. BAY11-7082 and TPCA-1), the corresponding inhibitors which were dissolved in dimethyl sulfoxide (DMSO) were added to the OGM composition at the corresponding increasing dosage concentrations: (BAY11-7082 at 5, 10, and 25 μM; TPCA-1 at 200, 400, and 1000 nM). The GM negative calcific controls and OGM positive calcification controls further were supplemented with DMSO as a vehicle control for possible perturbation due to DMSO exposure.

Alizarin red staining (ARS). Cell cultures cell cultures were fixed with a solution of 4% w/v paraformaldehyde (PFA) prepared in neutral saline solution for 1 hour at room temperature. Fixed cultures were then rinsed with de-ionized water and stained with 1% w/v Alizarin Red S solution prepared in de-ionized water with pH adjusted to 4.2 for 20 minutes at room temperature. Alizarin Red S staining is used to visualize calcific/mineral deposits generated by calcifying cells which retain red pigmentation. Stained samples were subsequently rinsed with de-ionized water to remove unbound dye, before subsequently imaging with a Zeiss stereoscopic dissection microscope outfitted with a high-resolution digital camera for images for analysis. ImageJ imaging analysis software with its inbuilt particle counting feature was used on monochromatic thresholded images to quantify the number of calcific lesion formed and average lesion size per field of view. Six independent biological samples with corresponding images were used for each condition.

### Example 9—. In Vitro Analysis of BAY11-7082 and TPCA-1

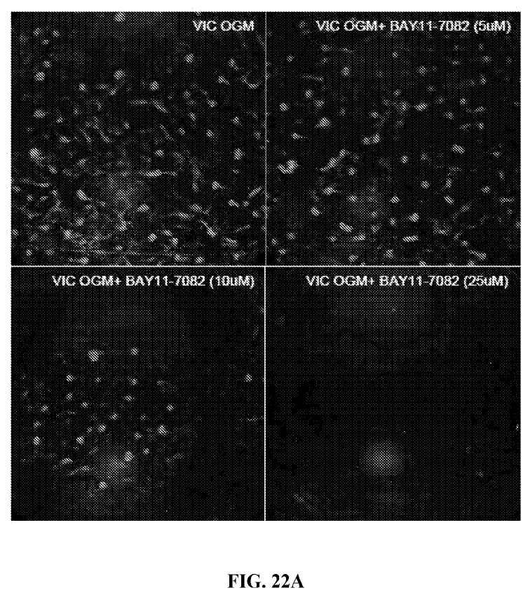

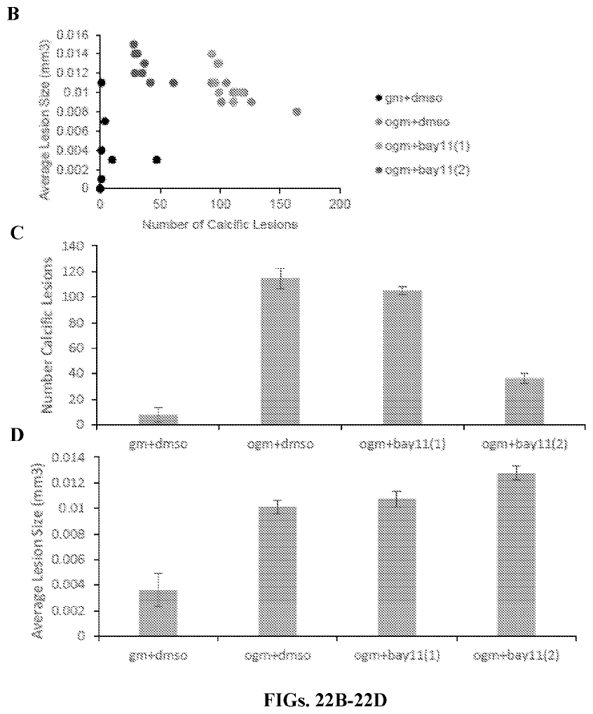

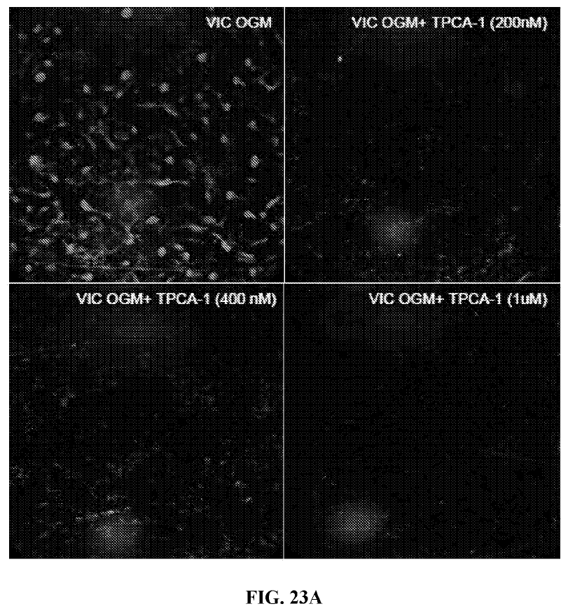

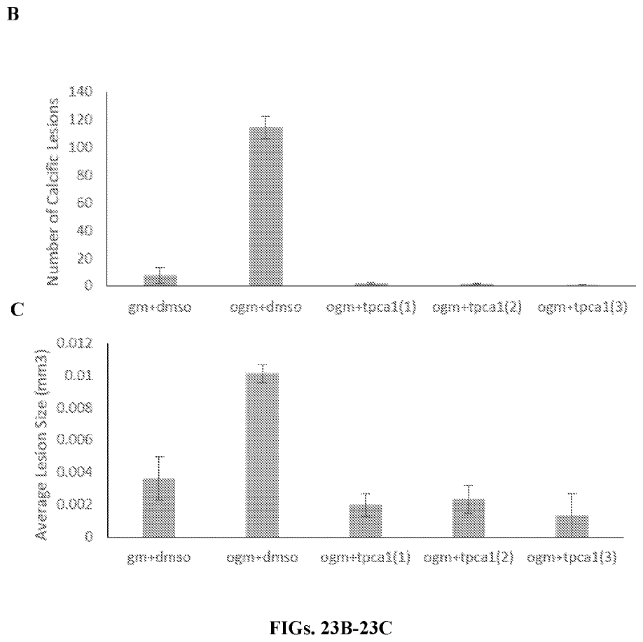

In this work, the NFκB pathway was intervened by administering either BAY11-7082 (FIGS. 22A-22D) or TPCA-1 (FIGS. 23A-23C) to calcifying valvular cell in vitro cultures every 48 hours for 14 days. Cellular calcific activity was measured by staining formed mineralized lesions by staining fixed cultures with Alizarin Red S and imaging samples for quantitative image analysis. BAY11-7082 is a potential anti-inflammatory agent that irreversibly inhibits and arrests IκB phosphorylation for subsequent ubiquination, thus maintaining the NFκB complex sequestered in the cellular cytoplasm in its inactive state. TPCA-1 is another potent and selective inhibitor, which targets IKKβ function in activating the NFκB complex for nuclear translocation and subsequent transcriptional activity.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed:

1. A method for inhibiting calcification of aortic valves or inhibiting aortic valve sclerosis/fibrosis, said method comprising:
selecting a subject having, or at risk of developing, calcific aortic valve disease or aortic valve sclerosis/fibrosis and
administering, to the selected subject, an inhibitor of the nuclear factor kappa B (NFκB) signaling pathway to inhibit calcification of the selected subject's aortic valves or aortic valve sclerosis/fibrosis.

2. The method of claim 1, wherein the selected subject has calcification of aortic valves.

3. The method of claim 1, wherein the selected subject has aortic valve sclerosis/fibrosis.

4. The method of claim 1, wherein the selected subject is at risk of developing calcific aortic valve disease.

5. The method of claim 1, wherein the selected subject is at risk of developing aortic valve sclerosis/fibrosis.

6. The method of claim 1, wherein the inhibitor of the NFκB signaling pathway is selected from the group consisting of a protein kinase inhibitor, a protein phosphatase inhibitor, an inhibitor of protein acetylation, a protein methyltransferase inhibitor, a proteasome inhibitor, an inhibitor of protein ubiquitination, an NFκB nuclear translocation inhibitor, an inhibitor of NFκB DNA binding activity, and an inhibitor of eNOS uncoupling.

7. The method of claim 6, wherein the inhibitor is a small molecule, a peptide, a nucleic acid, an antioxidant, a microbial protein, a viral protein, or an anti-inflammatory agent.

8. The method of claim 1, wherein the inhibitor of the NFκB signaling pathway is selected from the group consisting of ATP analogs, BMS-34554, parthenolide, arsenite, epoxyquinoids, gene-based inhibitors, SB203580, denbinobin, tyrosine kinase inhibitors, rhein, TNAP, betaine, epoxyquinol B, M2L, CCK-8, KSR2, golli BG21, BAY11-7082, protein phosphatase 2A, cytosine arabinoside, OspF, gallic acid, Daxx, anacardic acid, Set9 inhibitor, bortezomib, ALLnL, LLM, Z-LLnV, Z-LLL, lactacystine, N-cbz-Leu-Leu-leucinal (MG132), MG115, ubiquitin ligase inhibitors, salinosporamide A (NPI-0052), DCIC, TPCK, TLCK, BTEE, APNE, YopJ, R0196-9920, A20 (TNFAIP3), SN50, dehydroxymethylepoxyquinomicin, sesquiterpene lactones, decoy oligodeoxynucleotides, BH4, TPCA-1, and combinations thereof.

9. The method of claim 8, wherein the inhibitor of the NFκB signaling pathway is BAY11-7082.

10. The method of claim 8, wherein the inhibitor of the NFκB signaling pathway is BH4.

11. The method of claim 8, wherein the inhibitor of the NFκB signaling pathway is TPCA-1.

12. The method of claim 1, wherein the subject is a mammal, fish, bird, or reptile.

13. The method of claim 12, wherein the subject is a human, rabbit, cow, pig, sheep, chicken, rat, or mouse.

14. The method of claim 13, wherein the subject is a human.

15. A method for inhibiting myofibroblastic and osteogenic differentiation of aortic valvular interstitial cells (VIC) and/or aortic valvular endothelial cells (VEC), said method comprising:
administering, to the aortic valvular interstitial cells and/or aortic valvular endothelial cells, an inhibitor of the nuclear factor kappa B (NFκB) signaling pathway to inhibit myofibroblastic and osteogenic differentiation of the cells.

16. The method of claim 15, wherein the inhibitor of the NFκB signaling pathway is selected from the group consisting of a protein kinase inhibitor, a protein phosphatase inhibitor, an inhibitor of protein acetylation, a protein methyltransferase inhibitor, a proteasome inhibitor, an inhibitor of protein ubiquitination, an NFκB nuclear translocation inhibitor, an inhibitor of NFκB DNA binding activity, and an inhibitor of eNOS uncoupling.

17. The method of claim 16, wherein the inhibitor is a small molecule, a peptide, a nucleic acid, an antioxidant, a microbial protein, a viral protein, or an anti-inflammatory agent.

18. The method of claim 17, wherein the inhibitor of the NFκB signaling pathway is selected from the group consisting of ATP analogs, BMS-34554, parthenolide, arsenite, epoxyquinoids, gene-based inhibitors, SB203580, denbinobin, tyrosine kinase inhibitors, rhein, TNAP, betaine, epoxyquinol B, M2L, CCK-8, KSR2, golli BG21, BAY11-7082, protein phosphatase 2A, cytosine arabinoside, OspF, gallic acid, Daxx, anacardic acid, Set9 inhibitor, bortezomib, ALLnL, LLM, Z-LLnV, Z-LLL, lactacystine, N-cbz-Leu-Leu-leucinal (MG132), MG115, ubiquitin ligase inhibitors, salinosporamide A (NPI-0052), DCIC, TPCK, TLCK, BTEE, APNE, YopJ, R0196-9920, A20 (TNFAIP3), SN50, dehydroxymethylepoxyquinomicin, sesquiterpene lactones, decoy oligodeoxynucleotides, BH4, TPCA-1, and combinations thereof.

19. The method of claim 18, wherein the inhibitor of the NFκB signaling pathway is BAY11-7082.

20. The method of claim 18, wherein the inhibitor of the NFκB signaling pathway is BH4.

20. The method of claim 15, wherein said administering is carried out in vivo.

21. The method of claim 15, wherein said administering inhibits endothelial-to-mesenchymal transition in valvular endothelial cells.

22. The method of claim 14, wherein myofibroblastic and osteogenic differentiation of aortic valvular interstitial cells (VIC) is inhibited.

23. The method of claim 14, wherein myofibroblastic and osteogenic differentiation of aortic valvular endothelial cells (VEC) is inhibited.

24. The method of claim 14, wherein myofibroblastic and osteogenic differentiation of aortic valvular interstitial cells and aortic valvular endothelial cells is inhibited.
